SEQUENCE LISTING (196 sequences)

SEQ ID NO: 1 - protein, 24 aa, Conus ammiralis
  PEPTIDE (1)..(24): Xaa at residue 1 is Gln or pygro-Glu; Xaa at residues 7, 8 and 9 is Glu or gamma-carboxy-Glu; Xaa at residues 13 and 16 is Lys, nor-Lys, N-methyl-Lys, N,N-dimethyl-Lys or N,N, N-trimethyl-Lys
XGQDDSXXXDSQXVMXHGQRRERR

SEQ ID NO: 2 - protein, 17 aa, Conus betulinus
  PEPTIDE (1)..(17): Xaa at residues 3, 4, 7, 10 and 14 is Glu or gamma-carboxy-Glu; Xaa at residue 17 is Pro or hydroxyl-Pro
GGXXVRXSAXTLHXLTX

SEQ ID NO: 3 - protein, 17 aa, Conus betulinus
  PEPTIDE (1)..(17): Xaa at residues 3, 4, 7, 10 and 14 is Glu or gamma-carboxy-Glu; Xaa at residue 17 is Pro or hydroxy-Pro
GGXXVRXSAXTLHXITX

SEQ ID NO: 4 - protein, 17 aa, Conus betulinus
  PEPTIDE (1)..(17): Xaa at residues 3, 4, 7, 10 and 14 is Glu or gamma-carboxy-Glu; Xaa at residue 17 is Pro or hydroxy-Pro
DGXXVRXAAXTLNXLTX

SEQ ID NO: 5 - protein, 18 aa, Conus betulinus
  PEPTIDE (1)..(18): Xaa at residues 3, 7, 10, 14, 16 and 17 is Glu or gamma-carboxy-Glu
GYXDDRXIAXTVRXLXXA

SEQ ID NO: 6 - protein, 17 aa, Conus betulinus
  PEPTIDE (1)..(17): Xaa at residues 4, 7, 10 and 14 is Glu or gamma-carboxy-Glu; Xaa at residue 17 is Pro or hydroxy-Pro
GGGXVRXSAXTLHXITX

SEQ ID NO: 7 - protein, 18 aa, Conus bullatus
  PEPTIDE (1)..(18): Xaa at residue 2 is Pro or hydroxy-Pro; Xaa at residues 3, 7, 10,14, 16 and 17 is Glu or gamma-carboxy-Glu; Xaa at residue 5 is Tyr, mono-halo-Tyr, di-halo-Tyr, 125I-Tyr, O-sulpho-Tyr, O-phospho-Tyr or nitro-Tyr
NXXTXIXIVXISRXLXXI

SEQ ID NO: 8 - protein, 20 aa, Conus bullatus
  PEPTIDE (1)..(20): Xaa at residue 2 is Pro or hydroxy-Pro; Xaa at residues 3, 6, 9, 12, 16, 18 and 19 is Glu or gamma-carboxy- Glu; Xaa at residue 5 is Tyr, mono-halo-Tyr, di-halo-Tyr, 125I- Tyr, O-sulpho-Tyr, O-phos pho-Tyr or nitro-Tyr
NXXTXXNLXLVXISRXLXXI

SEQ ID NO: 9 - protein, 19 aa, Conus catus
  PEPTIDE (1)..(19): Xaa at residues 3, 4, 8, 11, 15 and 17 is Glu or gamma-carboxy-Glu
SDXXLLRXDVXTVLXLXRN

SEQ ID NO: 10 - protein, 19 aa, Conus catus
  PEPTIDE (1)..(19): Xaa at residues 3, 4, 8, 11, 15 and 17 is Glu or gamma-carboxy-Glu
GDXXLLRXDVXTVLXLXRD

SEQ ID NO: 11 - protein, 19 aa, Conus catus
  PEPTIDE (1)..(19): Xaa at residues 3, 4, 8, 11, 15 and 17 is Glu or gamma-carboxy-Glu
SDXXLLRXDVXTVLXPXRN

SEQ ID NO: 12 - protein, 17 aa, Conus catus
  PEPTIDE (1)..(17): Xaa at residues 2, 3, 7, 10 and 14 is Glu or gamma-carboxy-Glu
IXXGLIXDLXTARXRDS

SEQ ID NO: 13 - protein, 17 aa, Conus catus
  PEPTIDE (1)..(17): Xaa at residues 2, 3, 7, 10 and 14 is Glu or gamma-carboxy-Glu
IXXGLIXDLXAARXRDS

SEQ ID NO: 14 - protein, 29 aa, Conus catus
  PEPTIDE (1)..(29): Xaa at residues 2, 4, 10 and 16 is Glu or gamma-carboxy-Glu; Xaa at residues 3. 9, 25 and 28 is Pro or hydroxy-Pro; Xaa at residue 26 is Trp (D or L) or halo-Trp (D or L)
GXXXVGSIXXAVRQQXCIRNNNNRXXCXX

SEQ ID NO: 15 - protein, 17 aa, Conus distans
  PEPTIDE (1)..(17): Xaa at residues 5, 6, 8 and 12 is Glu or gamma-carboxy-Glu
TITAXXAXRTSXRMSSM

SEQ ID NO: 16 - protein, 19 aa, Conus distans
  PEPTIDE (1)..(19): Xaa at residue 1 is Gln or pyro-Glu; Xaa at residues 2, 7, 8, 10 and 14 is Glu or gamma-carboxy-Glu; Xaa at residues 4 and 6 is Pro or hydroxy-Pro
XXTXTXXXVXRHTXRLKSM

SEQ ID NO: 17 - protein, 15 aa, Conus episcopatus
  PEPTIDE (1)..(15): Xaa at residues 7, 11 and 13 is Glu or gamma- carboxy-Glu; Xaa at residue 14 is Lys, nor-Lys, N-methyl-Lys, N, N-dimethyl-Lys or N,N,N-trimethyl-Lys
GGKDIVXTITXLXXI

SEQ ID NO: 18 - protein, 19 aa, Conus figulinus
  PEPTIDE (1)..(19): Xaa at residues 2, 3, 4, 7, 11 and 15 is Glu or gamma-carboxy-Glu
GXXXVAXMAAXIARXNQAN

SEQ ID NO: 19 - protein, 18 aa, Conus figulinus
  PEPTIDE (1)..(18): Xaa at residue 2 is Tyr, mono-halo-Tyr, di- halo-Tyr, 125I-Tyr, O-sulpho-Tyr, O-phospho-Tyr or nitro-Tyr Xaa at residues 3, 7, 10, 14 and 17 is Glu or gamma-carboxy-Glu
SXXQARXVQXAVNXLXXR

SEQ ID NO: 20 - protein, 34 aa, Conus figulinus
  PEPTIDE (1)..(34): Xaa at residue 2 is Tyr, mono-halo-Tyr, di- halo-Tyr, 125I-Tyr, O-sulpho-Tyr, O-phospho-Tyr or nitro-Tyr Xaa at residues 3, 7, 10, 14 and 17 is Glu or gamma-carboxy-Glu; Xaa at residue 28 is Pro or hydroxy-Pro
SXXQARXVQXAVNXLXXRGXXIIMLGVXRDTRQF

SEQ ID NO: 21 - protein, 17 aa, Conus figulinus
  PEPTIDE (1)..(17): Xaa at residue 2 is Tyr, mono-halo-Tyr, di- halo-Tyr, 125I-Tyr, O-sulpho-Tyr, O-phospho-Tyr or nitro-Tyr; Xaa at residues 3, 7, 10, 14, 16 and 17 is Glu or gamma-carboxy- Glu
XXDDRXIAXTVRXLXXI

SEQ ID NO: 22 - protein, 19 aa, Conus figulinus
  PEPTIDE (1)..(19): Xaa at residues 5, 6, 9, 12 and 16 is Glu or gamma-carboxy-Glu
GNTAXXVRXAAXTLHXLSL

SEQ ID NO: 23 - protein, 23 aa, Conus figulinus
  PEPTIDE (1)..(23): Xaa at residues 8, 12, 15, 19 and 22 is Glu or gamma-carboxy-Glu
GSISMGFXHRRXIAXLVRXLAXI

SEQ ID NO: 24 - protein, 19 aa, Conus lynceus
  PEPTIDE (1)..(19): Xaa at residues 2, 3, 4, 7, 11 and 15 is Glu or gamma-carboxy-Glu
GXXXVAXMAAXIARXNAAN

SEQ ID NO: 25 - protein, 18 aa, Conus lynceus
  PEPTIDE (1)..(18): Xaa at residue 2 is Lys, nor-Lys, N-methyl- Lys, N,N-dimethyl-Lys or N,N,N-trimethyl-Lys; Xaa at residues 3, 4, 7, 10, 14, 16 and 17 is Glu or gamma-carboxy-Glu
GXXXDRXIVXTVRXLXXI

SEQ ID NO: 26 - protein, 19 aa, Conus lynceus
  PEPTIDE (1)..(19): Xaa at residues 2, 3, 4, 7, 11, 15 and 16 is Glu or gamma-carboxy-Glu; Xaa at residue 19 is Lys, nor-Lys, N-methyl-Lys, N,N-dimethyl-Lys or N,N,N-trimethyl-Lys
GXXXVAXMAAXLTRXXAVX

SEQ ID NO: 27 - protein, 24 aa, Conus purpurascens
  PEPTIDE (1)..(24): Xaa at residues 2, 3, 4, 10, 14 and 23 is Glu or gamma-carboxy-Glu; Xaa at residues 7 and 19 is Lys, nor-Lys, N-methyl-Lys, N,N-dimethyl-Lys or N,N,N-trimethyl-Lys
GXXXHSXXQXCLRXVRVNXVQQXC

SEQ ID NO: 28 - protein, 24 aa, Conus purpurascens
  PEPTIDE (1)..(24): Xaa at residues 2, 3, 4, 10, 14 and 23 is Glu or gamma-carboxy-Glu; Xaa at residues 7 is Lys, nor-Lys, N-methyl-Lys, N,N-dimethyl-Lys or N,N,N-trimethyl-Lys
GXXXHSXXQXCLRXVRVNNVQQXC

SEQ ID NO: 29 - protein, 24 aa, Conus purpurascens
  PEPTIDE (1)..(24): Xaa at residues 2, 3, 4, 10, 14 and 23 is Glu or gamma-carboxy-Glu; Xaa at residues 7 and 19 is Lys, nor-Lys, N-methyl-Lys, N,N-dimethyl-Lys or N,N,N-trimethyl-Lys
GXXXHSXXQXCLRXIRVNXVQQXC

SEQ ID NO: 30 - protein, 24 aa, Conus purpurascens
  PEPTIDE (1)..(24): Xaa at residues 2, 4, 10, 14 and 23 is Glu or gamma-carboxy-Glu; Xaa at residues 19 is Lys, nor-Lys, N-methyl- Lys, N,N-dimethyl-Lys or N,N,N-trimethyl-Lys
GXAXHXAFQXCLRXINVNXVQQXC

SEQ ID NO: 31 - protein, 15 aa, Conus purpurascens
  PEPTIDE (1)..(15): Xaa at residues 3, 4, 7, 10, 13 and 14 is Glu or gamma-carboxy-Glu
GLXXDIXFIXTIXXI

SEQ ID NO: 32 - protein, 15 aa, Conus stercusmuscarum
  PEPTIDE (1)..(15): Xaa at residues 3, 7, 13 and 14 is Glu or gamma-carboxy-Glu; Xaa at residue 11 is Lys, nor-Lys, N-methyl- Lys, N,N-dimethyl-Lys or N,N,N-trimethyl-Lys
ITXTDIXLVMXLXXI

SEQ ID NO: 33 - protein, 20 aa, Conus aurisiacus
  PEPTIDE (1)..(20): Xaa at residues 2, 4, 11 and 15 is Glu or gamma-carboxy-Glu; Xaa at residue 20 is Lys, nor-Lys, N-methyl- Lys, N,N-dimethyl-Lys or N,N,N-trimethyl-Lys
GXDXVSQMSXXILRXLELQX

SEQ ID NO: 34 - DNA, 31 bp, Artificial
  oligonucleotide primer
caggatcctgtatctgctggtgcccctggtg

SEQ ID NO: 35 - DNA, 23 bp, Artificial
  oligonucletide primer
aagctcgagtaacaacgcagagt

SEQ ID NO: 36 - DNA, 432 bp, Conus catus
gcgatgcaactgtacacgtatctgtatctgctggtgcccctggtgaccttccacctaatcctaggcacgggcacactagatcatggaggcgcactgactgaacgccgttcgggtgacgccacagcgctgagacctgagcctgtcctcctgcagaaatccgctgcccgcagcaccgacgacagtggcaaggacaggttgactcagatgaagaggattctcaaaaagcaaggaaacacggctaaaagcgacgaagagctactacgagaggatgtagagactgttttagaactcgaaaggaatggaaaaagataatcaagctgagtgttccacgtgacactcgtcagttctaaagtccccagataaatcgttccctattttgccacattctttctttctcttttcatttaattccccaaatctttcatgtttatt

SEQ ID NO: 37 - protein, 102 aa, Conus catus
MQLYTYLYLLVPLVTFHLILGTGTLDHGGALTERRSGDATALRPEPVLLQKSAARSTDDSGKDRLTQMKRILKKQGNTAKSDEELLREDVETVLELERNGKR

SEQ ID NO: 38 - protein, 19 aa, Conus catus
  PEPTIDE (1)..(19): Xaa at residues 3, 4, 8, 11, 15 and 17 is Glu or gamma-carboxy-Glu
SDXXLLRXDVXTVLXLXRN

SEQ ID NO: 39 - DNA, 432 bp, Conus catus
gcgatgcaactgtacacgtatctgtatctgctggtgcccctggtgaccttccacctaatcctaggcacgggcacactagatcatggaggcgcactgactgaacgccgttcgggtgacgccacagcgctgagacctgagcctgtcctcctgcagaaatccgctgcccgcagcaccgacgacagtggcaaggacaggttgactcagatgaagaggattctcaaaaagcaaggaaacacggctaaaggcgacgaagagctactacgagaggatgtagagactgttttagaactcgaaagggatggaaaaagataatcaagctgagtgttccacgtggcactcgtcagttctaaagtccccagataaatcgttccctattttgccacattctttctttctcttttcatttaattccccaaatctttcatgtttatt

SEQ ID NO: 40 - protein, 102 aa, Conus catus
MQLYTYLYLLVPLVTFHLILGTGTLDHGGALTERRSGDATALRPEPVLLQKSAARSTDDSGKDRLTQMKRILKKQGNTAKGDEELLREDVETVLELERDGKR

SEQ ID NO: 41 - protein, 19 aa, Conus catus
  PEPTIDE (1)..(19): Xaa at residues 3, 4, 8, 11, 15 and 17 is Glu or gamma-carboxy-Glu
GDXXLLRXDVXTVLXLXRD

SEQ ID NO: 42 - DNA, 432 bp, Conus catus
gcgatgcaactgtacacgtatctgtatctgctggcgcccctggtgaccttccacctaatcctaggcacgggcacactagatcatggaggcgcactgactgaacgccgttcgggtgacgccacagcgctgagacctgagcctgtcctcctgcagaaatccgctgcccgcagcaccgacgacagtggcaaggacaggttgactcagatgaagaggattctcaaaaagcaaggaaacacggctaaaagcgacgaagagctactacgagaggatgtagagactgttttagaacccgaaaggaatggaaaaagataatcaagctgagtgttccacgtgacactcgtcagttctaaagtccccagataaatcgttccctattttgccacattctttctttctcttttcatttaattccccaaatctttcatgtttatt

SEQ ID NO: 43 - protein, 102 aa, Conus catus
MQLYTYLYLLAPLVTFHLILGTGTLDHGGALTERRSGDATALRPEPVLLQKSAARSTDDSGKDRLTQMKRILKKQGNTAKSDEELLREDVETVLEPERNGKR

SEQ ID NO: 44 - protein, 19 aa, Conus catus
  PEPTIDE (1)..(19): Xaa at residues 3, 4, 8, 11, 15 and 17 is Glu or gamma-carboxy-Glu; Xaa at residue 16 is Pro or hydroxy-Pro
SDXXLLRXDVXTVLXXXRN

SEQ ID NO: 45 - DNA, 427 bp, Conus catus
gcgatgcaactgtacacgtatctgtatctgctggtgtccctggtgaccttccacctaatcctaggcacgggcacactagatcatggaggcgcactgactgaacgccgtttggctgacgccacagcgctggaagctgagcctgtcctcctgcagaaatccgctgcccgcagcaccgacaacaatggcaaggacaggtcgactcagatgaggaggattctcaaaaagcaaggaaacacggctagaatcgaggaaggtctgatagaggatctggagaccgctagagaacgcgacagtggaaaaagataatcaagctgagtgttccacgtgacactcatcagttctaaagtccccagataaatcgttccctatttttgccacattctttcttcctcttttcgtttaattccccaaatctttcatgtttatt

SEQ ID NO: 46 - protein, 100 aa, Conus catus
MQLYTYLYLLVSLVTFHLILGTGTLDHGGALTERRLADATALEAEPVLLQKSAARSTDNNGKDRSTQMRRILKKQGNTARIEEGLIEDLETARERDSGKR

SEQ ID NO: 47 - protein, 17 aa, Conus catus
  PEPTIDE (1)..(17): Xaa at residues 2, 3, 7, 10 and 14 is Glu or gamma-carboxy-Glu
IXXGLIXDLXTARXRDS

SEQ ID NO: 48 - DNA, 427 bp, Conus catus
gcgatgcaactgtacacgtatctgtatctgctggtgtccctggtgaccttccacctaatcctaggcacgggcacactagatcatggaggcgcactgactgaacgccgtttggctgacgccacagcgctggaagctgagcctgtcctcctgcagaaatccgctgcccgcagcaccgacaacaatggcaaggacaggtcgactcagatgaggaggattctcaaaaagcaaggaaacacggctagaatcgaggaaggtctgatagaggatctggaggctgctagagaacgcgacagtggaaaaagataatcaagctgagtgttccacgtgacactcatcagttctaaagtccccagataaatcgttccctatttttgccacattctttcttcctcttttcgtttaattccccaaatctttcatgtttatt

SEQ ID NO: 49 - protein, 100 aa, Conus catus
MQLYTYLYLLVSLVTFHLILGTGTLDHGGALTERRLADATALEAEPVLLQKSAARSTDNNGKDRSTQMRRILKKQGNTARIEEGLIEDLEAARERDSGKR

SEQ ID NO: 50 - protein, 17 aa, Conus catus
  PEPTIDE (1)..(17): Xaa at residues 2, 3, 7, 10 and 14 is Glu or gamma-carboxy-Glu
IXXGLIXDLXAARXRDS

SEQ ID NO: 51 - DNA, 433 bp, Conus catus
gcgatgcaactgtacacgtatctgtatctgctggtgcccctggtgaccttccacctaatcctaggcacgggcacactagatcatggaggcgcactgactgaacgccgttcggctgacgccacagcgctgaaacctgagcctgtcctcctgcagaaatccgctgcccgcagcaccgacgacaatggcaaagacaggttgactcacatgaagaggattctcaaaaaacgagcaaacaaagccagaggcgaaccagaagttggaagcataccggaggcagtaagacaacaagaatgtataagaaataataataatcgaccttggtgtcccaagtgacactcgtcagttctaaagtctccagatagatcgttccctatttttgccacactctttctttctcttttcatttaagttccccaaatctttcatgtttatt

SEQ ID NO: 52 - protein, 107 aa, Conus catus
MQLYTYLYLLVPLVTFHLILGTGTLDHGGALTERRSADATALKPEPVLLQKSAARSTDDNGKDRLTHMKRILKKRANKREPEVGSIPEAVRQQECIRNNNNRPWCPK

SEQ ID NO: 53 - protein, 29 aa, Conus catus
  PEPTIDE (1)..(29): Xaa at residues 2, 4, 10 and 16 is Glu or gamma-carboxy-Glu; Xaa at residues 3, 9, 25 and 28 is Pro or hydroxy-Pro; Xaa at residue 26 is Tyr, mono-halo-Tyr, di-halo- Tyr, 125I-Tyr, O-sulpho-Tyr, O-phospho-Tyr or nitro-Tyr
GXXXVGSIXXAVRQQXCIRNNNNRXXCXK

SEQ ID NO: 54 - DNA, 430 bp, Conus bullatus
gcgatgcaactgtacacgtatctgtatctgctggtgcccttggtgaccttccacctaatcctgggcacgggcacactagatcatggaggcgcactgactgaacgccgttcggctgacgccacagcactgaaacctgagcctgtcctcctgcagaaaaccgctgcccgcagcaccgacgacaatggcaagaagaggctgactcagaggaagaggattctcaaaaagcgaggaaacacggctagaaaccccgaaacttatatagagattgtggagatttctagggaactcgaagagattggaaaaagataatcaagctgggtgttccacgtgacactcgtcagttctgaagtcccgaggtagatcgttccctatttttgccacactctttctttctcttttcatttaattccccaaatctttcatgtttatt

SEQ ID NO: 55 - protein, 101 aa, Conus bullatus
MQLYTYLYLLVPLVTFHLILGTGTLDHGGALTERRSADATALKPEPVLLQKTAARSTDDNGKKRLTQRKRILKKRGNTARNPETYIEIVEISRELEEIGKR

SEQ ID NO: 56 - protein, 18 aa, Conus bullatus
  PEPTIDE (1)..(18): Xaa at residue 1 is Pro or hydroxy-Pro; Xaa at residues 3, 5, 7, 10, 14, 16 and 17 is Glu or gamma-carboxy- Glu; Xaa at residue 5 is Tyr, mono-halo-Tyr, di-halo-Tyr, 125I- Tyr, O-sulpho-Tyr, O-phos pho-Tyr or nitro-Tyr
NXXTXIXIVXISRXLXXI

SEQ ID NO: 57 - DNA, 435 bp, Conus bullatus
gcgatgcaactgtacacgtatctgtatttgctggtgcccttggtgaccttccacctaatcctgggcacgggcacactagatcatggaggcgcactgactgaacgccgttcggctgacgccacagcgctgaaacctgagcctgtcctcctgcagaaaaccgctgcccgcagcaccgacgacaatggcaagaagaggctgactcagaggaagaggattctcaaaaagcgaggaaacacggctagaaaccccgaaacttattataatttagagcttgtggagatttctagggaactcgaagaaattggaaaaagataatcaagctgggtgttccacgtgacactcgtcagttcttaagtcccgaggtagatcgttccctatttttgccacactctttctttctcttttcatttaattccccaaactttcatgtttatt

SEQ ID NO: 58 - protein, 103 aa, Conus bullatus
MQLYTYLYLLVPLVTFHLILGTGTLDHGGALTERRSADATALKPEPVLLQKTAARSTDDNGKKRLTQRKRILKKRGNTARNPETYYNLELVEISRELEEIGKR

SEQ ID NO: 59 - protein, 20 aa, Conus bullatus
  PEPTIDE (1)..(20): Xaa at residue 1 is Pro or hydroxy-Pro; Xaa at residues 3, 9, 12, 16, 18 and 19 is Glu or gamma-carboxy-Glu; Xaa at residues 5 and 6 is Tyr, mono-halo-Tyr, di-halo-Tyr, 125I- Tyr, O-sulpho-Tyr, O-phospho-Tyr or nitro-Tyr
NXXTXXNLXLVXISRXLXXI

SEQ ID NO: 60 - DNA, 425 bp, Conus betulinus
gcgatgcaactgtacacgtatctgtatctgctggtgcccctggtgaccttctacctaatcctaggcacgggcacgctaggtcatggaggcgcactgactgaacgccgtttggctgatgccacagcgctgaaacctgagcctgtcctcctgcagaaatccgccgcccgcagcaccgacgacaatggcaaggacaggttgactcagatgatcaggattctcaaaaagcgaggaaacatggccagaggcggcgaagaagttagagagtctgcagagactcttcatgaactcacgccgtaggaaaaagaaaaagattaatcaagctgggtgtcccacgtgacactcgtcagttctaaagtccccagtttcctatctttgccacgtttctttttcttttcattcaattccccaaatctttcatgtttatt

SEQ ID NO: 61 - protein, 95 aa, Conus betulinus
MQLYTYLYLLVPLVTFYLILGTGTLGHGGALTERRLADATALKPEPVLLQKSAARSTDDNGKDRLTQMIRILKKRGNMRGEEVRESAETLHELTP

SEQ ID NO: 62 - protein, 17 aa, Conus betulinus
  PEPTIDE (1)..(17): Xaa at residues 3, 4, 7, 10 and 14 is Glu or gamma-carboxy-Glu; X aa at residue 17 is Pro or hydroxy-Pro
GGXXVRXSAXTLHXLTX

SEQ ID NO: 63 - DNA, 425 bp, Conus betulinus
gcgatgcaactgtatacgtatctgtatctgctggtgccgctggtgaccttctacctaatcctaggcacgggcacgctaggtcatggaggcgcactgactgaacgccgtttggctgacgccacagcgctgaaacctgagcctgtcctcctgcagaaatccgccgcccgcagcactgacgacaatggcaaggacaggttgactcagatgatcaggattctcaaaaagcgaggaaacatggccagaggcggcgaagaagttagagagtctgcagagactcttcatgaaatcacgccgtaggaaaaagaaaaagattaatcaagctgggtgttccacgtgacactcgccagttctaaagtccccagtttcctatctttgccaggtttctttctcttttcattcaattccccaaatctttcatgtttatt

SEQ ID NO: 64 - protein, 95 aa, Conus betulinus
MQLYTYLYLLVPLVTFYLILGTGTLGHGGALTERRLADATALKPEPVLLQKSAARSTDDNGKDRLTQMIRILKKRGNMRGEEVRESAETLHEITP

SEQ ID NO: 65 - protein, 17 aa, Conus betulinus
  PEPTIDE (1)..(17): Xaa at residues 3, 4, 7, 10 and 14 is Glu or gamma-carboxy-Glu; Xaa at residue 17 is Pro or hydroxy-Pro
GGXXVRXSAXTLHXITX

SEQ ID NO: 66 - DNA, 425 bp, Conus betulinus
gcgatgcaactgtacacgtatctgtatctgctggtgcccctggtgaccttctacctaatcctaggcacgggcacgctaggtcatggaggcgcactgactgaacgccgtttggctgacgccacagcgctgaaacctaagcctatcctcctgcagaaatccgccgcccgcagcactgacgacaatggcaaggacaggttgactcagatgatcaggattctcaaaaagcgaggaaacatgggcagagacggcgaagaagtcagagaggctgcagagactcttaatgaactcacgccgtaggaaaaagaaaaagattaatcaagctgggtgttccacgtgacactcgtcagttctaaagtacccagtttcctatctttgccacgtttctttttctttccattcaattccccaaatctttcatgtttatt

SEQ ID NO: 67 - protein, 97 aa, Conus betulinus
MQLYTYLYLLVPLVTFYLILGTGTLGHGGALTERRLADATALKPKPILLQKSAARSTDDNGKDRLTQMIRILKKRGNMGRDGEEVREAAETLNELTP

SEQ ID NO: 68 - protein, 17 aa, Conus betulinus
  PEPTIDE (1)..(17): Xaa at residues 3, 4, 7, 10 and 14 is Glu or gamma-carboxy-Glu; Xaa at residue 17 is Pro or hydroxy-Pro
DGXXVRXAAXTLNXLTX

SEQ ID NO: 69 - DNA, 437 bp, Conus betulinus
gcgatgcaactgtacacgtatctgtatctgctggtgcccctggtgaccttccacctaatcctaggcacgggcacgctaggtcatggaggcgcactgactgaaagccgttcggctgacgccacagcactgaaaccagggcctgtcctcctgcagaaatccgctgcccgcagcaccgacgacaatggcaaggacaggttgactcagatgaagaggactctcaaaaagcgaggaaacacggccagaggctacgaagatgatagagagattgcagagactgttagagaactcgaggaagcaggaaaatgaaaaagattaatcaagctgggtgttccacgtgacacttgtcagttctaaagtccccagatagatcgttccctatttttgccacattctttttttctcttttcatttaattccccaaatctttcatgtttatt

SEQ ID NO: 70 - protein, 98 aa, Conus betulinus
MQLYTYLYLLVPLVTFHLILGTGTLGHGGALTESRSADATALKPGPVLLQKSAARSTDDNGKDRLTQMKRTLKKRGNTRYEDDREIAETVRELEEAGK

SEQ ID NO: 71 - protein, 18 aa, Conus betulinus
  PEPTIDE (1)..(18): Xaa at residue 2 is Tyr, mono-halo-Tyr, di-halo-Tyr, 125I-Tyr, O-sulpho-Tyr, O-phospho-Tyr or nitro-Tyr; Xaa at residue 3, 7, 10, 14, 16 and 17 is Glu or gamma-carboxy-Glu
GXXDDRXIAXTVRXLXXA

SEQ ID NO: 72 - DNA, 425 bp, Conus betulinus
gcgatgcaactgtacacgtatctgtatctgctggtgccgctggtgaccttctacctaatcctaggcacgggcacgctaggtcatggaggcgcactgactgaacgccgtttggctgacgccacagcgctgaaacctgagcctgtcctcctgcagaaatccgccgcccgcagcactgacgacaatggcaaggacaggttgactcagatgatcaggattctcaaaaagcgaggaaacatggccagaggcggcggagaagttagagagtctgcagagactcttcatgaaatcacgccgtaggaaaaagaaaaagattaatcaagctgggtgttccacgtgacactcgtcagttctaaagtccccagtttcctatctttgccaggtttctttctcttttcattcaattccccaaatctttcatgtttatt

SEQ ID NO: 73 - protein, 95 aa, Conus betulinus
MQLYTYLYLLVPLVTFYLILGTGTLGHGGALTERRLADATALKPEPVLLQKSAARSTDDNGKDRLTQMIRILKKRGNMRGGEVRESAETLHEITP

SEQ ID NO: 74 - protein, 17 aa, Conus betulinus
  PEPTIDE (1)..(17): Xaa at residues 4, 7, 10 and 14 is Glu or gamma-carboxy-Glu; Xaa at residue 17 is Pro or hydroxy-Pro
GGGXVRXSAXTLHXITX

SEQ ID NO: 75 - DNA, 434 bp, Conus ammiralis
gcgatgcaactgtacacgtatctgtgtctgctggtgcccctggtgaccttctacctaattctaggcacgggcacactagctcatggaggcgcactgaccgaacgccgtttggctcacgccagagtaatagaacctgatcctgcccccctggagaactccgctctccgcagcatccgacgacaacgacaaggacaggatgactcagaggaagaggattctcaaaaagtgatgaaacacggccagaggcgcgaaagaagatagaaataatgcggaggctgttagagaaagactcgaagaaataggaaaaaggtaatcaagctgggtgtttcacgtgacactcatcagttctaaagtccccagatagatcgttccctatttttgccatattctttccttctcttttcatgtaattccccaaatctttcatgtttatt

SEQ ID NO: 76 - protein, 85 aa, Conus ammiralis
MQLYTYLCLLVPLVTFYLILGTGTLAHGGALTERRLAHARVIEPDPAPLENSALRSIRRQRQGQDDSEEEDSQKVMKHGQRRERR

SEQ ID NO: 77 - protein, 24 aa, Conus ammiralis
  PEPTIDE (1)..(24): Xaa at residue 1 is Gln or pyro-Glu; Xaa at residues 7, 8, 9 and 22 is Glu or gamma-carboxy-Gl
XGQDDSXXXDSQKVMKHGQRRXRR

SEQ ID NO: 78 - DNA, 421 bp, Conus episcopatus
gcgatgcaactgtacacgtatctgtgtctgctggtgcccctggtgaccttctacctaattctaggcacgggcacactagctcatggaggcgcactgactgaacatcgttcggccgacgccacagcactgaaacctgagcctgtcctcctgcagaaatccgctgcccgcagcaccgacgacaacggcaaggacaggttgactcggtggaaggggattctcaaaaagcgaggaaacacggccagaggcgggaaagatattgtggagactattacagaactcgaaaaaataggaaaaaggtaatcaagctgggtgttccacgtgacactcatcagttctaaagtccccagatagatcgttccctatttttgccatattctttctttctcttttcatgtaattccccaaatctttcatgtttatt

SEQ ID NO: 79 - protein, 96 aa, Conus episcopatus
MQLYTYLCLLVPLVTFYLILGTGTLAHGGALTEHRSADATALKPEPVLLQKSAARSTDDNGKDRLTRWKGILKKRGNTRGKDIVETITELEKIGKR

SEQ ID NO: 80 - protein, 15 aa, Conus episcopatus
  PEPTIDE (1)..(15): Xaa at residues 7, 11 and 13 is Glu or gamma- carboxy-Glu
GGKDIVXTITXLXKI

SEQ ID NO: 81 - DNA, 433 bp, Conus lynceus
gcgatgcaactgtacacgtatctgtatctgctggtgcccctggtgaccttccacctaatcctaggcacgggcacactagatcatggaggcgcactgactgaacgccgttcgactgatgccatagcactgaaacctgagcctgtcctcctgcagaaatcctctgcccgcagcaccgacgataatggcaacgacaggttgactcagatgaagaggatcctcaaaaagcgaggaaacaaagccagaggcgaagaagaagttgcaaaaatggcggcagagattgccagagaaaacgctgcaaatgggaaatgataatcaagttgggtgttccacgtgacactcgtcagttctaaagtccccagatagatcgttccctatttttgccacattctttctttctcttttcatttaattccccaaatctttcatgtttatt

SEQ ID NO: 82 - protein, 99 aa, Conus lynceus
MQLYTYLYLLVPLVTFHLILGTGTLDHGGALTERRSTDAIALKPEPVLLQKSSARSTDDNGNDRLTQMKRILKKRGNKREEEVAKMAAEIARENAANGK

SEQ ID NO: 83 - protein, 19 aa, Conus figulinus
  PEPTIDE (1)..(19): Xaa at residues 2, 3, 4, 11 and 15 is Glu or gamma-carboxy-Glu
GXXXVAKMAAXIARXNAAN

SEQ ID NO: 84 - DNA, 430 bp, Conus lynceus
gcgatgcaactgtacacgtatctgtatctgctggtgcccctggtgatcttctacctaatcctaggcacgggcacgctaggtcatggaggcacactgactgaacgccgttcggctgatgccacagcactgaaacctgagcctgtcctcctgcagaaatccgctgcccgcagcaccggcgacgatgccaaggagaggttgactcagacgaagaggattcgcaaaaagcgagcaaacacgaccagaggcaaagaagaggatagagagattgtggagactgttagagaactcgaagaaataggaaaaagatgatcaagctgggtgttccacgtgacactcgtcagttccaaagtccccagatagatcgttccctatttttgccacattctttctttcttttttcatttaattccccaaatctttcatgtttatt

SEQ ID NO: 85 - protein, 101 aa, Conus lynceus
MQLYTYLYLLVPLVIFYLILGTGTLGHGGTLTERRSADATALKPEPVLLQKSAARSTGDDAKERLTQTKRIRKKRANTTRGKEEDREIVETVRELEEIGKR

SEQ ID NO: 86 - protein, 18 aa, Conus lynceus
  PEPTIDE (1)..(18): Xaa at residues 3, 4, 7, 10, 14, 16 and 17 is Glu or gamma-carbox y-Gl
GKXXDRXIVXTVRXLXXI

SEQ ID NO: 87 - DNA, 433 bp, Conus lynceus
gcgatgcaactgtacacgtatctgtatctgctggtgcccctggtgaccttccacctaatcctaggcacgggcacactagatcatggaggcgcactgactgaacgccgttcgactgacgccatagcactgaaacctgagcctgtcctcctgcagaaatcctctgcccgcagcaccgacgacaatggcaacgacaggttgattcagatgaagaggattctcaaaaagcgaggaaacaaagccagaggcgaagaggaagttgcaaaaatggcggcagagcttaccagagaagaagctgtaaaggggaaatgataatcaagttgggtgttccacgtgacactcgtcagttctaaagtccccagatagatcgttccctatttttgccacattctttctttctattttcatttaattccccaaatctttcatgtttatt

SEQ ID NO: 88 - protein, 99 aa, Conus lynceus
MQLYTYLYLLVPLVTFHLILGTGTLDHGGALTERRSTDAIALKPEPVLLQKSSARSTDDNGNDRLIQMKRILKKRGNKREEEVAKMAAELTREEAVKGK

SEQ ID NO: 89 - protein, 19 aa, Conus lynceus
  PEPTIDE (1)..(19): Xaa at residues 2, 3, 4, 11, 15 and 16 is Glu or gamma-carboxy-Gl
GXXXVAKMAAXLTRXXAVK

SEQ ID NO: 90 - DNA, 433 bp, Conus figulinus
gcgatgcaactgtacacgtatctgtatctgctggtgcccctggtgaccttctacctaatcctaggcacgggcacgctaggtcatggaggcgcactgactgaacgccgtttggctgacgccacagcgctgaaacctgagcctgtcctcctgcagaaatccgctgcccgcagcaccgacgacaatgacaaggacaggctgacccagatgaagaggattttcaaaaagcgaggaaacaaagccagaggcgaggaagaagttgcagagatggcggcagagattgcaagagaaaatcaagcaaacgggaaaagataatcaaactgggtgttccacgtgacactcgtcagttctaaagtccccagataggtcgttctctatgtttgccacattctttctttttcttttcatttaattccccaaatctttcatgtttatt

SEQ ID NO: 91 - protein, 100 aa, Conus figulinus
MQLYTYLYLLVPLVTFYLILGTGTLGHGGALTERRLADATALKPEPVLLQKSAARSTDDNDKDRLTQMKRIFKKRGNKREEEVAEMAAEIARENQANGKR

SEQ ID NO: 92 - protein, 19 aa, Conus figulinus
  PEPTIDE (1)..(19): Xaa at residues 2, 3, 4, 7, 11 and 15 is Glu or gamma-carboxy-Glu
GXXXVAXMAAXIARXNQAN

SEQ ID NO: 93 - DNA, 431 bp, Conus figulinus
gcgatgcaactgtacacgtatctgtatctgctggtgcccctggtgaccttctacctaatcctagggacgggcacactagctcatggaggcgcaccgactgaacgccgtttggctgacaccacagcactgaaacccgagcatgtcctcctgcagatgtccgctgcccgcagcaccaacgataatggcaaggacaggttgactcagatgaagaggattctcaaaaagcaaggaaacacagccagaagctacgaacaagctagagaagttcaggaggctgttaatgaactcaaggaaagaggtaaaaagataatcatgctgggtgttccacgtgacactcgtcagttctaaagcccccagatagattgttccgtatttttaccacgttctttctttctcttttcatttaattccccaaatctttcatgtttatt

SEQ ID NO: 94 - protein, 114 aa, Conus figulinus
MQLYTYLYLLVPLVTFYLILGTGTLAHGGAPTERRLADTTALKPEHVLLQMSAARSTNDNGKDRLTQMKRILKKQGNTARSYEQAREVQEAVNELKERGKKIIMLGVPRDTRQF

SEQ ID NO: 95 - protein, 18 aa, Conus figulinus
  PEPTIDE (1)..(18): Xaa at residue 2 is Tyr, mono-halo-Tyr, di- halo-Tyr, 125I-Tyr, O-sulpho-Tyr, O-phospho-Tyr or nitro-Tyr; Xaa at residues 3, 7, 10, 14 and 17 is Glu or gamma-carboxy-Glu
SXXQARXVQXAVNXLKXR

SEQ ID NO: 96 - DNA, 431 bp, Conus figulinus; identical to SEQ ID NO: 93

SEQ ID NO: 97 - protein, 114 aa, Conus figulinus; identical to SEQ ID NO: 94

SEQ ID NO: 98 - protein, 34 aa, Conus figulinus
  PEPTIDE (1)..(34): Xaa at residue 2 is Tyr, mono-halo-Tyr, di-halo-Tyr, 125I-Tyr, O-sulpho-Tyr, O-phospho-Tyr or nitro-Tyr; Xaa at residues 3, 7, 10, 14 and 17 is Glu or gamma-carboxy-Glu; Xaa at residue 28 is Pro or hydroxy-Pro
SXXQARXVQXAVNXLKXRGKKIIMLGVXRDTRQF

SEQ ID NO: 99 - DNA, 429 bp, Conus figulinus
gcgatgcaactgtacacgtatctgtatctgctggtgcccctggtgacgttccacctaatcctaggcacgggcacactagctcatggaggcgcactggctgaacgccgtttggctgacgccacagcgctgaaacctgagcctgtcctcctgcagaaatccgctgcccgcagcaccgacgacaatggcaaggacaggttgactgagatgaagaggattctcaaaaagcgaggaaacacggccagagactacgaagatgatagagagattgcagagactgttagagaactcgaagaaataggtaaaagataatcaagctgggtgttcaattgacactcatcagttctaaagtccccagatagatcgttccctaattttgccacgttctttctttctcttttcatttaattccccaaatctttcatgtttatt

SEQ ID NO: 100 - protein, 99 aa, Conus figulinus
MQLYTYLYLLVPLVTFHLILGTGTLAHGGAERRLADATALKPEPVLLQKSAARSTDDNGKDRLTEMKRILKKRGNTARDYEDDREIAETVRELEEIGKR

SEQ ID NO: 101 - protein, 18 aa, Conus figulinus
  PEPTIDE (1)..(18): Xaa at residue 2 is Tyr, mono-halo-Tyr, di- halo-Tyr, 125I-Tyr, O-sulpho-Tyr, O-phospho-Tyr or nitro-Tyr; Xaa at residues 3, 7, 10, 14, 16 and 17 is Glu or gamma-carboxy- Glu
DXXDDRXIAXTVRXLXXI

SEQ ID NO: 102 - DNA, 419 bp, Conus figulinus
gcgatgcaactgtacacgtatctgtatctgctggtgcccctggtgaccttctacctaatcctaggcacgggcacgctaggtcatggaggcgcactgactgaacgccgtttggctgacgccacagcgctgaaacctgagcctgtcctcctgcagaaatccgctgcccgcagcaccgacgacaatggcaaggacaggttgactcagatgaaggggactgtcaaaaagcgaggaaacacggccgaagaagttagagaggctgcagagactcttcatgaactctcgctgtaggaaaaagaaaaagattaatcaagctgggtgttccacgtgacactcgtcagttctaaagtccccagttccctatctttgccacgttttttctttctcttttcatccaattccccaaatctttcatgtttatt

SEQ ID NO: 103 - protein, 94 aa, Conus figulinus
MQLYTYLYLLVPLVTFYLILGTGTLGHGGALTERRLADATALKPEPVLLQKSAARSTDDNGKDRLTQMKGTVKKRGNTAEEVREAAETLHELSL

SEQ ID NO: 104 - protein, 19 aa, Conus figulinus
  PEPTIDE (1)..(19): Xaa at residues 5, 6, 9, 12 and 16 is Glu or gamma-carboxy-Glu
GNTAXXVRXAAXTLHXLSL

SEQ ID NO: 105 - DNA, 427 bp, Conus figulinus
gcgatgcaactgtacacgtatctgtatctgctggtgcctctggtgaccttccacctaatcctaggcacgggcacactaggtcatggaggcgcactgactgaacgccgtttggctgacgccacagcgctgaaacctgagcctgtcctcctgcagaaatccgctgcccgcagcaccgacgtcaatggcaaggacaggttgactgagatgaagaggattctcaaaaagcgaggaagcatatccatgggcttcgaacatagaagagagattgcagagttggttagagaactcgctgaaataggtaaacgataatcaagctgggtgttccactaacactcgtcagttctaaagtccccagatagatcgttccctatctttgccacattttttttctcttttcatttaattccccaaatctttcatgtttatt

SEQ ID NO: 106 - protein, 101 aa, Conus figulinus
MQLYTYLYLLVPLVTFHLILGTGTLGHGGALTERRLADATALKPEPVLLQKSAARSTDVNGKDRLTEMKRILKKRGSISMGFEHRREIAELVRELAEIGKR

SEQ ID NO: 107 - protein, 23 aa, Conus figulinus
  PEPTIDE (1)..(23): Xaa at residues 8, 12, 15, 19 and 22 is Glu or gamma-carboxy-Glu
GSISMGFXHRRXIAXLVRXLAXI

SEQ ID NO: 108 - DNA, 427 bp, Conus distans
gcgatgcaactgtacacgtatctgtatctgctggtgcccctggtggccttccacctaatccaaggcacgggcacactaggccatggaggcgcactgactgaaggccgttcggctgacgccacagcgccgaaacctgaacctgtcctcctgcagaaatccgatgcccgcagcgccgacgacaacggcaaggacaagttgactcagatgaagaggactctgaaaaagcaaggacacattgccagaaccataactgctgaagaggcagagaggactagtgaaagaatgtcatcaatgggaaaaagataatcaagctgggtgttccacgtgacactcgtcagttctaaagtccccagataaatcgttccctgtttttgccctgttctttctttctcttttcattcaattccccaaatctttcatgtttatt

SEQ ID NO: 109 - protein, 98 aa, Conus distans
MQLYTYLYLLVPLVAFHLIQGTGTLGHGGALTEGRSADATAPKPEPVLLQKSDARSADDNGKDKLTQMKRTLKKQGHIARTITAEEAERTSMSSMGKR

SEQ ID NO: 110 - protein, 17 aa, Conus distans
  PEPTIDE (1)..(17): Xaa at residues 5, 6, 8 and 12 is Glu or gamma-carboxy-Glu
TITAXXAXRTSXRMSSM

SEQ ID NO: 111 - DNA, 415 bp, Conus distans
gcgatgcaactgtacacgtatctgtatctgctggtatccctggtggccttccacctaatccaaggaacgggcacgctaggccatggaggcgcactgactgaaggccgttcggctgacgccacagcgccgaaacctgaacctgtgctcgtgcagaaatcggatgcccgcagcgccgacgacaaccgcaaggacaagttgactcagatgaagaggattctgaaaaagcaagaaaccccaactcctgaagaggtagagcgccataccgaaagactcaaaagcatgggaaaaagataatcaagctgggtgttccacgtgacactcgtcagttctaaagtccccagatggatcgttccctgtttttgccccgttctttcgttctcttttcattcaattccccaaatctttcatgtttatt

SEQ ID NO: 112 - protein, 96 aa, Conus distans
MQLYTYLYLLVSLVAFHLIQGTGTLGHGGALTEGRSADATAPKPEPVLVQKSDARSADDNRKDKLTQMKRILKKQETPTPEEVERHTERLKSMGKR

SEQ ID NO: 113 - protein, 19 aa, Conus distans
  PEPTIDE (1)..(19): Xaa at residue 1 is Gln or pyro-Glu; Xaa at residues 2, 4, 6, 7, 8, 10 and 14 is Glu or gamma-carboxy-Glu
XXTXTXXXVXRHTXRLKSM

SEQ ID NO: 114 - DNA, 439 bp, Conus purpurascens
gcgatgcaactgtacacgtatctgtatctgctggtgcccctggtgaccttccacctaatcctaggcacgggaatgctagctcatggagacacactgactgaacgccgttcggttgacgccacagcactgaaacctgagcctgtcctcctgcagaaatccgctgcccgcagcaccgacgacaatgacaaggacaggttgactcagatgaagaggattctcaaaaagcgaggaaacaaagccagaggcgaagaagaacattccaagtatcaagagtgtcttagagaagtaagagtaaataaggtacaacaagaatgttaatcaagctgggtgttccacgtgacactcgtcagttctaaagtccccagatagatcgttcccgatttttgccacattctttctttctcttttcatttaattccccaaatctttcatgtttatt

SEQ ID NO: 115 - protein, 102 aa, Conus purpurascens
MQLYTYLYLLVPLVTFHLILGTGMLAHGDTLTERRSVDATALKPEPVLLQKSAARSTDDNDKDRLTQMKRILKKRGNKREEEHSKYQECLREVRVNKVQQEC

SEQ ID NO: 116 - protein, 24 aa, Conus purpurascens
  PEPTIDE (1)..(24): Xaa at residues 2, 3, 4, 10, 14 and 23 is Glu or gamma-carboxy-Glu; Xaa at residue 8 is Tyr, mono-halo-Tyr, di- halo-Tyr, 125I-Tyr,O-sulpho-Tyr, O-phospho-Tyr or nitro-Tyr
GXXXHSKXQXCLRXVRVNKVQQXC

SEQ ID NO: 117 - DNA, 436 bp, Conus purpurascens
gcgatgcaactgtacacgtatctgtatctgctggtgcccctggtgaccttccacctaatcctaggcacgggcacactagctcatggaggcgcactgactgaacgcggttccactgacgccacagcactgaaacctgagcctgtcctgcaggaatctgatgcccgcagcaccgacgacaatgacaaggacaggttgactcagatgaagaggattctcaaaaagcgaggaaacaaagccagaggcgaagaagaacattccaagtatcaggagtgtcttagagaagtaagagtaaataacgtacaacaagaatgttaatcaagctgggtgttccacgtgacactcgtcagttctaaagtccccagatagatcgttccctatttttgccacattctttctttctcttttcatttaattccccaaatctttcatgtttatt

SEQ ID NO: 118 - protein, 101 aa, Conus purpurascens
MQLYTYLYLLVPLVTFHLILGTGTLAHGGALTERGSTDATALKPEPVLQESDARSTDDNDKDRLTQMKRILKKRGNKREEEHSKYQECLREVRVNNVQQEC

SEQ ID NO: 119 - protein, 24 aa, Conus purpurascens
  PEPTIDE (1)..(24): Xaa at residues 2, 3, 4, 10, 14 and 23 is Glu or gamma-carboxy-Glu; Xaa at residue 8 is Tyr, mono-halo-Tyr, di-halo-Tyr, 125I-Tyr,O-sulpho-Tyr, O-phospho-Tyr or nitro-Tyr
GXXXHSKXQXCLRXVRVNNVQQXC

SEQ ID NO: 120 - DNA, 439 bp, Conus purpurascens
gcgatgcaactgtacacgtatctgtatctgctggtgcccctggtgaccttccacctaatcctaagcacgggcacactagctcatggaggcacactgactgaacgccgttcgactgacaccacagcactgaaacctgagcctgtcctcctgcagaaatctgatgcccgcagcaccgacgacaatgacaaggacaggttgactcagatgaagaggattctcaaaaagcgaggaaacaaagccagaggcgaagaagaacattccaagtatcaggagtgtcttagagaaataagagtaaataaggtacaacaagaatgttaatcaagctgggtgttccacgtgacacccgtcagttctaaagtccccagatagatcgttccctatttttgccacattctttctttctcttttcatttaattccccaaatctttcatgtttatt

SEQ ID NO: 121 - protein, 102 aa, Conus purpurascens
MQLYTYLYLLVPLVTFHLILSTGTLAHGGTLTERRSTDTTALKPEPVLLQKSDARSTDDNDKDRLTQMKRILKKRGNKREEEHSKYQECLREIRVNKVQQEC

SEQ ID NO: 122 - protein, 24 aa, Conus purpurascens
  PEPTIDE (1)..(24): Xaa at residues 2, 3, 4, 10, 14 and 23 is Glu or gamma-carboxy-Glu; Xaa at residue 8 is Tyr, mono-halo-Tyr, di- halo-Tyr, 125I-Tyr, O-sulpho-Tyr, O-phospho-Tyr or nitro-Tyr
GXXXHSKXQXCLRXIRVNKVQQXC

SEQ ID NO: 123 - DNA, 439 bp, Conus purpurascens
gcgatgcaactgtacacgtatctgtatctgctggtgcccctggtgaccttccacctaatcctaagcacgggcacactagctcatggagacacactgactgaacgccgttcggttgacgccacagcactgaaacctgagcctgtcctcctgcagaaatccgctgcccgcagcaccgacgacgatgacaaggacaggttgactcagaggaagaggattctcaaaaagcaaggaaacaaagccagaggcgaagcagaacattacgcgtttcaggagtgtcttagagaaataaatgtaaataaggtacaacaagaatgttaatcaagctgggtgttctacgtgacactcgtcagttctaaagtccccagatagatcgttccctatttttgccacattctttctttctcttttcatttaattccccaaatctttcatgtttatt

SEQ ID NO: 124 - protein, 102 aa, Conus purpurascens
MQLYTYLYLLVPLVTFHLILSTGTLAHGDTLTERRSVDATALKPEPVLLQKSAARSTDDDDKDRLTQRKRILKKQGNKREAEHYAFQECLREINVNKVQQEC

SEQ ID NO: 125 - protein, 24 aa, Conus purpurascens
  PEPTIDE (1)..(24): Xaa at residues 2, 4, 10, 14 and 23 is Glu or gamma-carboxy-Glu;Xaa at residue 6 is Tyr, mono-halo-Tyr, di- halo-Tyr, 125I-Tyr, O-sulpho-Tyr, O-phospho-Tyr or nitro-Tyr
GXAXHXAFQXCLRXINVNKVQQXC

SEQ ID NO: 126 - DNA, 421 bp, Conus purpurascens
gcgatgcaactgtacacgtatctgtatctgctggtgcccctggtgaccttccacctaatcctaggcacgggaatgctagctcatggagacacactgactgaacgccgttcggttgacgccacagcactgaaacctgagcctgtcctcctgcagaaatccgctgcccgcagcaccgacgccaatggcaaggacaggttgactcagaggaagaggattctcaaaaagcgaggaaacatggccaggggcttagaagaagatatagagtttattgagacgatcgaagaaattggaaaaagataaccaagctgggtgttccacgtgacactcgtcggttctaaagtccccagatagatcgttcactatttttgccacattctttctttctcttttcatttaattccccaaatctttcatgtttatt

SEQ ID NO: 127 - protein, 96 aa, Conus purpurascens
MQLYTYLYLLVPLVTFHLILGTGMLAHGDTLTERRSVDATALKPEPVLLQKSAARSTDANGKDRLTQRKRILKKRGNMRLEEDIEFIETIEEIGKR

SEQ ID NO: 128 - protein, 15 aa, Conus purpurascens
  PEPTIDE (1)..(15): Xaa at residues 3, 4, 7, 10, 13 and 14 is Glu or gamma-carboxy-Glu
GLXXDIXFIXTIXXI

SEQ ID NO: 129 - DNA, 418 bp, Conus stercusmuscarum
gcgatgcaactgtacacgtatctgtatctgctggtgcccctggtgaccttccacctaatcctgggcacgggcacactagatcatggaggcgcactgactgaacgccgttcggctgacgccacagcgctgaaacctgagcctgtcctgcagaaatccgctgccggcagcaccgacgacaacggcaaggacaggttgactcagatgaagaggattctcaaaaagcgaggaaacacggctagaatcaccgaaactgatatagagcttgttatgaaattagaagaaattggaaaaagataatcaagctgggtgttccacgtgacactcgtcagttctgaagtcccgaggtagatcgttccctatttttgccacattctttctttctcttttcatgtaattccccaaatctttcatgtttatt

SEQ ID NO: 130 - protein, 97 aa, Conus stercusmuscarum
MQLYTYLYLLVPLVTFHLILGTGTLDHGGALTERRSADATALKPEPVLQKSAAGSTDDNGKDRLTQMKRILKKRGNTARITETDIELVMKLEEIGKR

SEQ ID NO: 131 - protein, 15 aa, Conus stercusmuscarum
  PEPTIDE (1)..(15): Xaa at residues 3, 7, 13 and 14 is Glu or gamma-carboxy-Glu
ITXTDIXLVMKLXXI

SEQ ID NO: 132 - protein, 17 aa, Conus geographus
  PEPTIDE (1)..(17): Xaa is Glu or gamma-carboxy-Glu
GEXXLQXNQXLIRXKSN

SEQ ID NO: 133 - protein, 24 aa, Conus ammiralis
  PEPTIDE (1)..(24): Xaa is Glu or gamma-carboxy-Glu
ZGQDDSEXXDSQKVMKHGQRRERR

SEQ ID NO: 134 - protein, 17 aa, Conus betulinus
  PEPTIDE (1)..(17): Xaa is Glu or gamma-carboxy-Glu
GGXXVRXSAXTLHXLTP

SEQ ID NO: 135 - protein, 17 aa, Conus betulinus
  PEPTIDE (1)..(17): Xaa is Glu or gamma-carboxy-Glu
GGXXVRXSAXTLHXITP

SEQ ID NO: 136 - protein, 17 aa, Conus betulinus
  PEPTIDE (1)..(17): Xaa is Glu or gamma-carboxy-Glu
DGXXVRXAAXTLNXLTP

SEQ ID NO: 137 - protein, 18 aa, Conus betulinus
  PEPTIDE (1)..(18): Xaa is Glu or gamma-carboxy-Glu
GYXDDRXIAXTVRXLEEA

SEQ ID NO: 138 - protein, 17 aa, Conus betulinus
  PEPTIDE (1)..(17): Xaa is Glu or gamma-carboxy-Glu
GGGXVRXSAXTLHXITP

SEQ ID NO: 139 - protein, 18 aa, Conus bullatus
  PEPTIDE (1)..(18): Xaa is Glu or gamma-carboxy-Glu
NPXTYIXIVXISRXLEEI

SEQ ID NO: 140 - protein, 20 aa, Conus bullatus
  PEPTIDE (1)..(20): Xaa is Glu or gamma-carboxy-Glu
NPXTYYNLXLVXISRELEEI

SEQ ID NO: 141 - protein, 19 aa, Conus catus
  PEPTIDE (1)..(19): Xaa is Glu or gamma-carboxy-Glu
SDXXLLRXDVXTVLXLERN

SEQ ID NO: 142 - protein, 19 aa, Conus catus
  PEPTIDE (1)..(19): Xaa is Glu or gamma-carboxy-Glu
GDXXLLRXDVXTVLXLERD

SEQ ID NO: 143 - protein, 19 aa, Conus catus
  PEPTIDE (1)..(19): Xaa is Glu or gamma-carboxy-Glu
SDXXLLRXDVXTVLXPERN

SEQ ID NO: 144 - protein, 17 aa, Conus catus
  PEPTIDE (1)..(17): Xaa is Glu or gamma-carboxy-Glu
IEXGLIXDLXTARXRDS

SEQ ID NO: 145 - protein, 15 aa, Conus catus
  PEPTIDE (1)..(15): Xaa is Glu or gamma-carboxy-Glu
IEXGLIXDLXRXRDS

SEQ ID NO: 146 - protein, 29 aa, Conus catus
  PEPTIDE (1)..(29): Xaa is Glu or gamma-carboxy-Glu
GEPXVGSIPXAVRQQECIRNNNNRPWCPK

SEQ ID NO: 147 - protein, 17 aa, Conus distans
  PEPTIDE (1)..(17): Xaa is Glu or gamma-carboxy-Glu
TITAXXAXRTSXRMSSM

SEQ ID NO: 148 - protein, 19 aa, Conus distans
  PEPTIDE (1)..(19): Xaa is Glu or gamma-carboxy-Glu
ZETPTPXXVXRHTXRLKSM

SEQ ID NO: 149 - protein, 15 aa, Conus episcopatus
  PEPTIDE (1)..(15): Xaa is Glu or gamma-carboxy-Glu
GGKDIVXTITXLXKI

SEQ ID NO: 150 - protein, 19 aa, Conus figulinus
  PEPTIDE (1)..(19): Xaa is Glu or gamma-carboxy-Glu
GEXXVAXMAAXIARXNQAN

SEQ ID NO: 151 - protein, 18 aa, Conus figulinus
  PEPTIDE (1)..(18): Xaa is Glu or gamma-carboxy-Glu
SYXQARXVQXAVNXLKER

SEQ ID NO: 152 - protein, 34 aa, Conus figulinus
  PEPTIDE (1)..(34): Xaa is Glu or gamma-carboxy-Glu
SYXQARXVQXAVNXLKERGKKIIMLGVPRDTRQF

SEQ ID NO: 153 - protein, 18 aa, Conus figulinus
  PEPTIDE (1)..(18): Xaa is Glu or gamma-carboxy-Glu
DYXDDRXIAXTVRXLEEI

SEQ ID NO: 154 - protein, 19 aa, Conus figulinus
  PEPTIDE (1)..(19): Xaa is Glu or gamma-carboxy-Glu
GNTAXXVRXAAXTLHELSL

SEQ ID NO: 155 - protein, 23 aa, Conus figulinus
  PEPTIDE (1)..(23): Xaa is Glu or gamma-carboxy-Glu
GSISMGFXHRRXIAXLVRELAEI

SEQ ID NO: 156 - protein, 19 aa, Conus lynceus
  PEPTIDE (1)..(19): Xaa is Glu or gamma-carboxy-Glu
GEXXVAKMAAXIARXNAAN

SEQ ID NO: 157 - protein, 18 aa, Conus lynceus
  PEPTIDE (1)..(18): Xaa is Glu or gamma-carboxy-Glu
GKXXDRXIVXTVRXLEEI

SEQ ID NO: 158 - protein, 19 aa, Conus lynceus
  PEPTIDE (1)..(19): Xaa is Glu or gamma-carboxy-Glu
GEXXVAKMAAXLTRXEAVK

SEQ ID NO: 159 - protein, 24 aa, Conus purpurascens
  PEPTIDE (1)..(24): Xaa is Glu or gamma-carboxy-Glu
GEXXHSKYQXCLRXVRVNKVQQEC

SEQ ID NO: 160 - protein, 24 aa, Conus purpurascens
  PEPTIDE (1)..(24): Xaa is Glu or gamma-carboxy-Glu
GEXXHSKYQXCLRXVRVNNVQQEC

SEQ ID NO: 161 - protein, 24 aa, Conus purpurascens
  PEPTIDE (1)..(24): Xaa is Glu or gamma-carboxy-Glu
GEXXHSKYQXCLRXIRVNKVQQEC

SEQ ID NO: 162 - protein, 24 aa, Conus purpurascens
  PEPTIDE (1)..(24): Xaa is Glu or gamma-carboxy-Glu
GEAXHYAFQXCLRXINVNKVQQEC

SEQ ID NO: 163 - protein, 15 aa, Conus purpurascens
  PEPTIDE (1)..(15): Xaa is Glu or gamma-carboxy-Glu
GLXXDIXFIXTIXEI

SEQ ID NO: 164 - protein, 15 aa, Conus stercusmuscarum
  PEPTIDE (1)..(15): Xaa is Glu or gamma-carboxy-Glu
ITXTDIXLVMKLXEI

SEQ ID NO: 165 - protein, 24 aa, Conus ammiralis
ZGQDDSEEEDSQKVMKHGQRRERR

SEQ ID NO: 166 - protein, 17 aa, Conus betulinus
GGEEVRESAETLHELTP

SEQ ID NO: 167 - protein, 17 aa, Conus betulinus
GGEEVRESAETLHEITP

SEQ ID NO: 168 - protein, 17 aa, Conus betulinus
DGEEVREAAETLNELTP

SEQ ID NO: 169 - protein, 18 aa, Conus betulinus
GYEDDREIAETVRELEEA

SEQ ID NO: 170 - protein, 17 aa, Conus betulinus
GGGEVRESAETLHEITP

SEQ ID NO: 171 - protein, 18 aa, Conus bullatus
NPETYIEIVEISRELEEI

SEQ ID NO: 172 - protein, 20 aa, Conus bullatus
NPETYYNLELVEISRELEEI

SEQ ID NO: 173 - protein, 19 aa, Conus catus
SDEELLREDVETVLELERN

SEQ ID NO: 174 - protein, 19 aa, Conus catus
GDEELLREDVETVLELERD

SEQ ID NO: 175 - protein, 19 aa, Conus catus
SDEELLREDVETVLEPERN

SEQ ID NO: 176 - protein, 17 aa, Conus catus
IEEGLIEDLETARERDS

SEQ ID NO: 177 - protein, 17 aa, Conus catus
IEEGLIEDLEAARERDS

SEQ ID NO: 178 - protein, 29 aa, Conus catus
GEPEVGSIPEAVRQQECIRNNNNRPWCPK

SEQ ID NO: 179 - protein, 15 aa, Conus distans
TITAEEAERTSMSSM

SEQ ID NO: 180 - protein, 19 aa, Conus distans
ZETPTPEEVERHTERLKSM

SEQ ID NO: 181 - protein, 15 aa, Conus episcopatus
GGKDIVETITELEKI

SEQ ID NO: 182 - protein, 19 aa, Conus figulinus
GEEEVAEMAAEIARENQAN

SEQ ID NO: 183 - protein, 18 aa, Conus figulinus
SYEQAREVQEAVNELKER

SEQ ID NO: 184 - protein, 34 aa, Conus figulinus
SYEQAREVQEAVNELKERGKKIIMLGVPRDTRQF

SEQ ID NO: 185 - protein, 18 aa, Conus figulinus
DYEDDREIAETVRELEEI

SEQ ID NO: 186 - protein, 19 aa, Conus figulinus
GNTAEEVREAAETLHELSL

SEQ ID NO: 187 - protein, 23 aa, Conus figulinus
GSISMGFEHRREIAELVRELAEI

SEQ ID NO: 188 - protein, 19 aa, Conus lynceus
GEEEVAKMAAEIARENAAN

SEQ ID NO: 189 - protein, 18 aa, Conus lynceus
GKEEDREIVETVRELEEI

SEQ ID NO: 190 - protein, 19 aa, Conus lynceus
GEEEVAKMAAELTREEAVK

SEQ ID NO: 191 - protein, 24 aa, Conus purpurascens
GEEEHSKYQECLREVRVNKVQQEC

SEQ ID NO: 192 - protein, 24 aa, Conus purpurascens
GEEEHSKYQECLREVRVNNVQQEC

SEQ ID NO: 193 - protein, 24 aa, Conus purpurascens
GEEEHSKYQECLREIRVNKVQQEC

SEQ ID NO: 194 - protein, 24 aa, Conus purpurascens
GEAEHYAFQECLREINVNKVQQEC

SEQ ID NO: 195 - protein, 15 aa, Conus purpurascens
GLEEDIEFIETIEEI

SEQ ID NO: 196 - protein, 15 aa, Conus stercusmuscarum
ITETDIELVMKLEEI

(19) United States
(12) Patent Application Publication
(10) Pub. No.: US 2006/0241046 A1
(43) Pub. Date: Oct. 26, 2006

(54) Linear gamma-carboxyglutamate rich conotoxins

(75) Inventors: Baldomero M. Olivera, Salt Lake City, UT (US); J. Michael McIntosh, Salt Lake City, UT (US); James E. Garrett, Salt Lake City, UT (US); Craig S. Walker, Salt Lake City, UT (US); Maren Watkins, Salt Lake City, UT (US); Robert M. Jones, Salt Laker City, UT (US)

Correspondence Address: ROTHWELL, FIGG, ERNST & MANBECK, P.C., 1425 K STREET, N.W., SUITE 800, WASHINGTON, DC, 20005, US

(73) Assignees: University of Utah Research Foundation, Salt Lake City, UT (US); Cognetix, Inc., Salt Lake City, UT (US)

(21) Appl. No.: 11/476,638

(22) Filed: Jun. 29, 2006

Related U.S. Application Data
(63) Continuation of application No. 11/018,369, filed on Dec. 22, 2004, now abandoned.
Application No. 11/018,369 is a continuation of application No. 10/092,367, filed on Mar. 7, 2002, now abandoned.
(60) Provisional application No. 60/273,639, filed on Mar. 7, 2001.

(51) Int. Cl. A61K 38/16; A61K 38/10; C07K 14/435

(52) U.S. Cl. 514/12; 514/13; 530/324; 530/326

(57) ABSTRACT

The invention relates to linear y-carboxyglutamate rich conotoxins, derivatives or pharmaceutically acceptable salts thereof, and uses thereof, including the treatment of neurologic and psychiatric disorders, such as anticonvulsant agents, as neuroprotective agents or for the management of pain. The invention further relates to nucleic acid sequences encoding the conopeptides and encoding propeptides, as well as the propeptides.

### REFERENCE TO RELATED APPLICATION

The present application is a continuation of U.S. patent application Ser. No. 11/018,369 filed 22 Dec. 2004, which in turn is a continuation of U.S. patent application Ser. No. 10/092,367 filed 7 Mar. 2002. Ser. No. 10,092,367 is related to and claims priority under 35 USC §119(e) to U.S. provisional patent application Ser. No. 60/273,639 filed 7 Mar. 2001. Each application is incorporated herein by reference.

This invention was made with Government support under Grant No. PO1 GM48677 awarded by the National Institute of General Medical Sciences, National Institutes of Health, Bethesda, Md. The United States Government has certain rights in the invention.

### BACKGROUND OF THE INVENTION

The invention relates to linear y-carboxyglutamate rich conotoxins, derivatives or pharmaceutically acceptable salts thereof, and uses thereof, including the treatment of neurologic and psychiatric disorders, such as anticonvulsant agents, as neuroprotective agents or for the management of pain. The invention further relates to nucleic acid sequences encoding the conopeptides and encoding propeptides, as well as the propeptides.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Conus is a genus of predatory marine gastropods (snails) which envenomate their prey. Venomous cone snails use a highly developed projectile apparatus to deliver their cocktail of toxic conotoxins into their prey. In fish-eating species such as Conus magus the cone detects the presence of the fish using chemosensors in its siphon and when close enough extends its proboscis and fires a hollow harpoon-like tooth containing venom into the fish. The venom immobilizes the fish and enables the cone snail to wind it into its mouth via an attached filament. For general information on Conus and their venom see the website address http://grimwade.biochem.unimelb.edu.au/cone/referenc.html. Prey capture is accomplished through a sophisticated arsenal of peptides which target specific ion channel and receptor subtypes. Each Conus species venom appears to contain a unique set of 50-200 peptides. The composition of the venom differs greatly between species and between individual snails within each species, each optimally evolved to paralyse it's prey. The active components of the venom are small peptides toxins, typically 12-30 amino acid residues in length and are typically highly constrained peptides due to their high density of disulphide bonds.

The venoms consist of a large number of different peptide components that when separated exhibit a range of biological activities: when injected into mice they elicit a range of physiological responses from shaking to depression. The paralytic components of the venom that have been the focus of recent investigation are the α-, ω- and μ-conotoxins. All of these conotoxins act by preventing neuronal communication, but each targets a different aspect of the process to achieve this. The α-conotoxins target nicotinic ligand gated channels, the i-conotoxins target the voltage-gated sodium channels and the ω-conotoxins target the voltage-gated calcium channels (Olivera et al., 1985; Olivera et al., 1990). For example a linkage has been established between α-, αA- & φ-conotoxins and the nicotinic ligand-gated ion channel; ω-conotoxins and the voltage-gated calcium channel; μ-conotoxins and the voltage-gated sodium channel; δ-conotoxins and the voltage-gated sodium channel; κ-conotoxins and the voltage-gated potassium channel; conantokins and the ligand-gated glutamate (NMDA) channel.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by a specific disulfide bond frameworks combined with hypervariable amino acids within disulfide loops (for a review see McIntosh et al., 1998).

The conantokins are structurally unique. In contrast to the well characterized conotoxins from Conus venoms, most conantokins do not contain disulfide bonds. However, they contain 4-5 residues of the unusual modified amino acid γ-carboxyglutamic acid. The occurrence of this modified amino acid, which is derived post-translationally from glutamate in a vitamin K-dependent reaction, was unprecedented in a neuropeptide. It has been established that the conantokins have N-methyl-D-aspartate (NMDA) antagonist activity, and consequently target the NMDA receptor. The conantokins reduce glutamate (or NMDA) mediated increases in intracellular Ca²⁺ and cGMP without affecting kainate-mediated events (Chandler et al., 1993). Although these peptides have actions through polyamine responses of the NMDA receptors, the neurochemical profile of these polypeptides is distinct from previously described noncompetitive NMDA antagonists (Skolnick et al., 1992).

Ischemic damage to the central nervous system (CNS) may result form either global or focal ischemic conditions. Global ischemia occurs under conditions in which blood flow to the entire brain ceases for a period of time, such as may result from cardiac arrest. Focal ischemia occurs under conditions in which a portion of the brain is deprived of its normal blood supply, such as may result from thromboembolytic occlusion of a cerebral vessel, traumatic head or spinal cord injury, edema or brain or spinal cord tumors. Both global and focal ischemic conditions have the potential for widespread neuronal damage, even if the global ischemic condition is transient or the focal condition affects a very limited area.

Epilepsy is a recurrent paroxysmal disorder of cerebral function characterized by sudden brief attacks of altered consciousness, motor activity, sensory phenomena or inappropriate behavior caused by abnormal excessive discharge of cerebral neurons. Convulsive seizures, the most common form of attacks, begin with loss of consciousness and motor control, and tonic or clonic jerking of all extremities but any recurrent seizure pattern may be termed epilepsy. The term primary or idiopathic epilepsy denotes those cases where no cause for the seizures can be identified. Secondary or symptomatic epilepsy designates the disorder when it is associated with such factors as trauma, neoplasm, infection, developmental abnormalities, cerebrovascular disease, or various metabolic conditions. Epileptic seizures are classified as partial seizures (focal, local seizures) or generalized seizures (convulsive or nonconvulsive). Classes of partial seizures include simple partial seizures, complex partial seizures and partial seizures secondarily generalized. Classes of generalized seizures include absence seizures, atypical absence seizures, myoclonic seizures, clonic seizures, tonic seizures, tonic-clonic seizures (grand mal) and atonic seizures. Therapeutics having anticonvulsant properties are used in the treatment of seizures. Most therapeutics used to abolish or attenuate seizures act at least through effects that reduce the spread of excitation from seizure foci and prevent detonation and disruption of function of normal aggregates of neurons. Traditional anticonvulsants that have been utilized include phenytoin, phenobarbital, primidone, carbamazepine, ethosuximnide, clonazepam and valproate. Several novel and chemically diverse anticonvulsant medications recently have been approved for marketing, including lamotrigine, ferlbamate, gabapentin and topiramate. For further details of seizures and their therapy, see Rall & Schleifer (1985) and The Merck Manual (1992).

(S)-Glutamic acid (Glu), which is the main excitatory neurotransmitter in the CNS, and other excitatory amino acids (EAA) operate through four different classes of receptors. In addition to the three heterogeneous classes of ionotropic EAA receptors (mGluRs), named M-methyl-D-aspartate (NMDA), (RS)-2-amino-3-(hydroxy-5-methyl-4-isoxazolyl)-propionic acid (AMPA) and Kainate (KA) receptors, a heterogeneous class of G-protein coupled EAA receptors (mGluRs) has been shown to have important functions in neuronal signalling processes. It is now generally agreed that iGluRs as well as mGluRs play important roles in the healthy as well as the diseased CNS, and that all subtypes of these receptors are potential targets for therapeutic intervention in a number of diseases. For a review, see Brauner-Osborne et al. (2000). 100131 The cloning of the different subunits of the iGluRs and of the eight subtypes of mGluRs represents a major breakthrough. Whereas at present six NMDA receptor subunits (NR1, NR2A-NR2D, and NR3A) have been cloned and characterised in regards to primary structure, four AMPA subunits (iGluR1-4) have similarly been characterized, and so far 5 subunits building blocks for KA-preferred receptors (iGluR5-7, KA1, and KA2) have been identified. Most if not all physiological iGluRs have heterotetra- or penatmeric structures, but the number of functional NMDA, AMPA, and KA receptors in the CNS is not known. At present 8 subtypes of the 7TM mGluRs have been characterized, but there is evidence to suggest that further subtypes of mGluRs may be identified. The structurally unique linear conantokin peptides disclosed in this patent represent a series of ligands capable of activating, blocking or allostericaly modulating both iGluRs and mGluRs—they represent essential pharmacological tools and potential therapeutics for treatment brain injury, stroke, Huntingdons disease, Parkinsons disease, Alzheimers disease, ALS, Epilepsy, Schizophrenia, pain, anxiety, AIDS related dementia, spinal injury amongst other chronic and acute diseases and conditions.

For example, the NMDA receptor is involved in a broad spectrum of CNS disorders. For example, during brain ischemia caused by stroke or traumatic injury, excessive amounts of the excitatory amino acid glutamate are released from damaged or oxygen deprived neurons. This excess glutamate binds the NMDA receptor which opens the ligand-gated ion channel thereby allowing Ca²⁺ influx producing a high level of intracellular Ca²⁺, which activates biochemical cascades resulting in protein, DNA and membrane degradation leading to cell death. This phenomenon, known as excitotoxicity, is also thought to be responsible for the neurological damage associated with other disorders ranging from hypoglycemia and cardiac arrest to epilepsy. In addition, there are reports indicating similar involvement in the chronic neurodegeneration of Huntington's, Parkinson's and Alzheimer's diseases.

Parkinson's disease is a progressive, neurodegenerative disorder. The etiology of the disorder is unknown in most cases, but has been hypothesized to involve oxidative stress. The underlying neuropathology in Parkinsonian patients is an extensive degenerations of the pigmented dopamine neurons in the substantia nigra. These neurons normally innervate the caudate and putamen nuclei. Their degeneration results in a marked loss of the neurotransmitter dopamine in the caudate and putamen nuclei. This loss of dopamine and its regulation of neurons in the caudate-putamen leads to the bradykinesia, rigidity, and tremor that are the hallmarks of Parkinson's disease. An animal model has been developed for Parkinson's disease (Zigmond et al., 1987) and has been used to test agents for anti-Parkinsonian activity (Ungerstedt et al., 1973).

The dopamine precursor, L-Dopa, is the current therapy of choice in treating the symptoms of Parkinson's disease. However, significant side effects develop with continued use of this drug and with disease progression, making the development of novel therapies important. Recently, antagonists of the NMDA subtype of glutamate receptor have been proposed as potential anti-Parkinsonian agents. (Borman, 1989; Greenamyre and O'Brien, 1991; Olney et al., 1987). In addition, antagonists of NMDA receptors potentiate the behavioral effects of L-Dopa and D1 dopamine receptor stimulation in animal models of Parkinson's disease. (Starr, 1995). These data suggest that NMDA receptor antagonists may be useful adjuncts to L-Dopa therapy in Parkinson's disease by decreasing the amount of L-Dopa required and thereby reducing undesirable side effects. In addition, antagonists of NMDA receptors have been shown to attenuate free radical mediated neuronal death. Thus, NMDA receptor antagonists may also prevent further degeneration of dopamine neurons in addition to providing symptomatic relief. Finally, NMDA receptor antagonists have been shown to potentiate the contralateral rotations induced by L-Dopa or D1 dopamine receptor antagonists in the animal model.

Pain, and particularly, persistent pain, is a complex phenomenon involving many interacting components. Numerous studies, however, have demonstrated a role for NMDA receptors in mediating persistent pain, and further that NMDA antagonists are effective in animal models of persistent pain. See foe example, PCT published application WO 98/03189.

Neuropsychiatric involvement of the NMDA receptor has also been recognized. Blockage of the NMDA receptor Ca²⁺ channel by the animal anesthetic phencyclidine produces a psychotic state in humans similar to schizophrenia (Johnson et al., 1990). Further, NMDA receptors have also been implicated in certain types of spatial learning (Bliss et al., 1993). In addition, numerous studies have demonstrated a role for NMDA receptors in phenomena associated with addiction to and compulsive use of drugs or ethanol. Furthermore, antagonists of NMDA receptors may be useful for treating addiction-related phenomena such as tolerance, sensitization, physical dependence and craving (for review see, Popik et al., 1995; Spanagel and Zieglgansberger, 1997; Trujillo and Akil, 1995).

There are several lines of evidence which suggest that NMDA antagonists may be useful in the treatment of HIV infection. First, the levels of the neurotoxin and NMDA agonist quinolinic acid are elevated in the cerebrospinal fluid of HIV-positive subjects (Heyes et al., 1989) and in murine retrovirus-induced immunodeficiency syndrome (Sei et al., 1996). Second, the envelope glycoprotein of HIV-1 alter NMDA receptor function (Sweetnam et al., 1993). Thirdly, NMDA antagonists can reduce the effects and neurotoxicity of GP-120 (Muller et al., 1996; Raber et al., 1996; Nishida et al., 1996). Fourth, GP-120 and glutamate act synergistically to produce toxicity in vitro (Lipton et al., 1991). And finally, memantine, an NMDA antagonist, protects against HIV infection in glial cells in vitro (Rytik et al., 1991). For a review of the use of NMDA antagonists in treating HIV infection, see Lipton (1994; 1996).

PCT published application WO 98/03189 has shown that the class of conopeptides termed conantokins are useful for treating each of the previously discussed disorders as well as several others, including mood disorders, urinary incontinence, dystonia and sleep disorders among others. U.S. Pat. No. 5,844,077 also discloses the use of conantokins for inducing analgesia and for neuroprotection.

It is desired to identify additional compounds which are useful as anticonvulsant, neuropretective, neuropsychiatric or analgesic agents.

### SUMMARY OF THE INVENTION

The present invention is directed to linear γ-carboxyglutamate rich conotoxins, derivatives or pharmaceutically acceptable salts thereof, and uses thereof, including the treatment of neurologic and psychiatric disorders, such as anticonvulsant agents, as neuroprotective agents or for the management of pain. The invention is further directed to nucleic acid sequences encoding the conopeptides and encoding propeptides, as well as the propeptides.

More specifically, the present invention is directed to linear γ-carboxyglutamate rich conotoxins, having the amino acid sequences:

| | | |
|---|---|---|
| Conotoxin-Af6: | X₆GQDDSX₁X₁X₁DSQX₂VMX₂HGQRRERR\hat{ } | (SEQ ID NO:1) |
| Conotoxin-Bt1: | GGX₁X₁VRX₁SAX₁TLHX₁LTX₅\hat{ } | (SEQ ID NO:2) |
| Conotoxin-Bt2: | GGX₁X₁VRX₁SAX₁TLHX₁ITX₅\hat{ } | (SEQ ID NO:3) |
| Conotoxin-Bt3: | DGX₁X₁VRX₁AAX₁TLNX₁LTX₅\hat{ } | (SEQ ID NO:4) |
| Conotoxin-Bt4: | GYX₁DDRX₁IAX₁TVRX₁LX₁X₁A# | (SEQ ID NO:5) |
| Conotoxin-Bt5: | GGGX₁VRX₁SAX₁TLHX₁ITX₅\hat{ } | (SEQ ID NO:6) |
| Conotoxin-Bu1: | NX₅X₁TX₃IX₁IVX₁ISRX₁LX₁X₁I# | (SEQ ID NO:7) |
| Conotoxin-Bu2: | NX₅XITX₃X₃NLX₁LVX₁ISRX₁LX₁X₁I# | (SEQ ID NO:8) |
| Conotoxin-C1: | SDX₁X₁LLRX₁DVX₁TVLX₁LX₁RN# | (SEQ ID NO:9) |
| Conotoxin-C2: | GDX₁X₁LLRX₁DVX₁TVLX₁LX₁RD# | (SEQ ID NO:10) |
| Conotoxin-C3: | SDX₁X₁LLRX₁DVX₁TVLX₁PX₁RN# | (SEQ ID NO:11) |
| Conotoxin-C4: | IX₁X₁GLIX₁DLX₁TARX₁RDS# | (SEQ ID NO:12) |
| Conotoxin-C5: | IX₁X₁GLIX₁DLX₁AARX₁RDS# | (SEQ ID NO:13) |
| Conotoxin-C6: | GX₁X₅X₁VGSIX₅X₁AVRQQX₁CIRNNNNRX5X₄CX5X2\hat{ } | (SEQ ID NO:14) |
| Conotoxin-Di1: | TITAX₁X₁AX₁RTSX₁RMSSM# | (SEQ ID NO:15) |
| Conotoxin-Di2: | X₆X₁TX₅TX₅X₁X₁VX₁RHTX₁RLKSM# | (SEQ ID NO:16) |
| Conotoxin-Ep1: | GGKDIVX₁TITX₁LX₁X₂I# | (SEQ ID NO:17) |
| Conotoxin-Fi1: | GX₁X₁X₁VAX₁MAAX₁IARX₁NQAN# | (SEQ ID NO:18) |
| Conotoxin-Fi2: | SX₃X₁QARX₁VQX₁AVNX₁LX₂X₁R# | (SEQ ID NO:19) |
| Conotoxin-Fi2a: | SX₃X₁QARX₁VQX₁AVNX₁LX₂X₁RGX2X2IIMLGVX5RDTRQF\hat{ } | (SEQ ID NO:20) |
| Conotoxin-Fi3: | D X₃X₁DDRX₁IAX₁TVRX₁LX₁X₁I# | (SEQ ID NO:21) |
| Conotoxin-Fi4: | GNTAX₁X₁VRX₁AAX₁TLHX₁LSL\hat{ } | (SEQ ID NO:22) |
| Conotoxin-Fi5: | GSISMGFX₁HRRX₁IAX₁LVRX₁LAX₁I# | (SEQ ID NO:23) |
| Conotoxin-L1: | GX₁X₁X₁VAX₁MAAX₁IARX₁NAAN# | (SEQ ID NO:24) |
| Conotoxin-L2: | GX₂X₁X₁DRX₁IVX₁TVRX₁LX₁X1I# | (SEQ ID NO:25) |
| Conotoxin-L3: | GX₁X₁X₁VAX₂MAAX₁LTRX₁X₁AVX2# | (SEQ ID NO:26) |
| Conotoxin-P1: | GX₁X₁X₁HSX₂X₃QX₁CLRX₁VRVNX2VQQX1C\hat{ } | (SEQ ID NO:27) |
| Conotoxin-P2: | GX₁X₁X₁HSX₂X₃QX₁CLRX₁VRVNNVQQX1C\hat{ } | (SEQ ID NO:28) |
| Conotoxin-P3: | GX₁X₁X₁HSX₂X₃QX₁CLRX₁IRVNX2VQQX1C\hat{ } | (SEQ ID NO:29) |
| Conotoxin-P4: | GX₁AX₁HX₃AFQX₁CLRX₁INVNX₂VQQX₁C\hat{ } | (SEQ ID NO:30) |
| Conotoxin-P5: | GLX₁X₁DIX₁FIX₁TIX₁X₁I# | (SEQ ID NO:31) |
| Conotoxin-Sm1: | ITX₁TDIX₁LVMX₂LX₁X₁I# | (SEQ ID NO:32) |

wherein X₁ is Glu or γ-carboxyglutamic acid (Gla); X₂ is Lys, nor-Lys, N-methyl-Lys, N,N-dimethyl-Lys or N,N,N-trimethyl-Lys; X₃ is Tyr, mono-halo-Tyr, di-halo-Tyr, O-sulpho-Tyr, O-phospho-Tyr or nitro-Tyr; X₄ is Trp (D or L) or halo-Trp (D or L); X₅ is Pro or hydroxy-Pro; and X₆ is Gln or pyroglutamate. The halo is preferably chlorine, bromine or iodine, more preferably iodine for Tyr and bromine for Trp. The C-terminus contains a carboxyl or an amide. The preferred C-terminus is shown herein in Tables 5 and 6, which shows an alignment of the conopeptides of the present invention.

The present invention is further directed to derivatives or pharmaceutically acceptable salts of the linear γ-carboxyglutamate rich conotoxins or their derivatives. Examples of derivatives include peptides in which the γ-carboxyglutamic acid at the X₁ residues of the peptides of the present invention other than those residues corresponding to residues 3 and 4 of conatntokin G, such as shown by the alignment set forth herein in Table 5 by X, is replaced by any other amino acids such that their NMDA antagonist activity is not adversely affected. Examples of such replacements include, but are not limited to Ser, Ala, Glu and Tyr. Other derivatives are produced by modification of the amino acids within the peptide structure. Modified amino acids include those which are described in Roberts et al. (1983). Other derivatives include peptides in which one or more residues have been deleted. It has been discovered that one to five of the C-terminal amino acid residues can be deleted without loss of activity. Substitutions of one amino acid for another can be made at one or more additional sites within the above peptide, and may be made to modulate one or more of the properties of the peptides. Substitutions of this kind are preferably conservative, i.e., one amino acid is replaced with one of similar shape and charge. Conservative substitutions are well known in the art and include, for example: alanine to glycine, arginine to lysine, asparagine to glutamine or histidine, glycine to proline, leucine to valine or isoleucine, serine to threonine, phenylalanine to tyrosine, and the like.

These derivatives also include peptides in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The Met residues may be substituted with norleucine (Nle). The aliphatic amino acids may be substituted by synthetic

Examples of synthetic aromatic amino acid include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1 -(2-pyrazolinyl)-Arg, 2-(4-piperinyl)-Gly, 2-(4-piperinyl)-Ala, 2-[3-(2S)pyrro-lininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference, and such as shown in the following schemes 1-3.

Optionally, in the linear γ-carboxyglutamate rich conotoxins of the present invention, the Asn residues may be modified to contain an N-glycan and the Ser, Thr and Hyp residues may be modified to contaian O-glycan (e.g., g-N, g-S, g-T and g-Hyp). In accordance with the present invention, a glycan shall mean any N—, S—or O-linked mono-, di-, tri-, poly- or oligosac-charide that can be attached to any hydroxy, amino or thiol group of natural or modified amino acids by synthetic or enzymatic methodologies known in the art. The monosaccharides making up the glycan can include D-allose, D-altrose, D-flucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose, These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2.5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GaINAc residue. The monosaccharide building blocks and the linkage attached to this first GalNAc residue define the “core glycans,” of which eight have been indentified. The type of glycosidic linkage (orientation and connectivities) are defined for each core glycan. Suitable glycans and glycan analogs are described further in U.S. Ser. No. 09/420,797 filed 19 Oct. 1999 and in PCT Application No. PCT/US99/24380 filed 19 Oct. 1999 (PCT Published Application No. WO 00/23092), each incorporated herein by reference. A preferred glycan is Gal(β1→3)GalNAc(α1→).

More specifically, the present invention is also directed to nucleic acids which encode linear γ-carboxyglutamate rich conotoxins of the present invention or which encodes precursor peptides for these conotoxins, as well as the precursor peptide. The nucleic acid sequences encoding the precursor peptides of conopeptides of the present invention are set forth in Table 4.

The present invention is further directed to uses of these peptides or nucleic acids as described herein including the treatment of neurologic and psychiatric disorders, such as anticonvulsant agents, as neuroprotective agents or for the management of pain.

### DETAILED DESCRIPTION OF THE PREFERRED EMBODIMENT

The present invention is directed to linear γ-carboxyglutamate rich conotoxins, derivatives or pharmaceutically acceptable salts thereof. The present invention is further directed to the use of this peptide, derivatives thereof and pharmaceutically acceptable salts thereof for the treatment of neurologic and psychiatric disorders, such as anticonvulsant agents, as neuroprotective agents or for the management of pain, e.g. as analgesic agents. Neurologic disorders and psychiatric disorders as used herein are intended to include such disorders as grouped together in The Merck Manual of Diagnosis and Therapy, inclusive of the disorders discussed in PCT published application WO 98/03189, incorporated herein by reference. The invention is further directed to nucleic acid sequences encoding the conopeptides and encoding propeptides, as well as the propeptides.

More specifically, the present invention is directed to the use of these compounds for the treatment and alleviation of epilepsy and as a general anticonvulsant agent. The present invention is also directed to the use of these compounds for reducing neurotoxic injury associated with conditions of hypoxia, anoxia or ischemia which typically follows stroke, cerebrovascular accident, brain or spinal cord trauma, myocardial infarct, physical trauma, drowning, suffocation, perinatal asphyxia, or hypoglycemic events. The present invention is further directed to the use of these compounds for treating neurodegeneration associated with Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia and neuronal damage associated with uncontrolled seizures. The present invention is also directed to the use of these compounds for treating chemical toxicity, such as addiction, drug craving, alcohol abuse, morphine tolerance, opioid tolerance and barbiturate tolerance. The present invention is further directed to treating psychiatric disorders, such as anxiety, major depression, manic-depressive illness, obsessive-compulsive disorder, schizophrenia and mood disorders (such as bipolar disorder, unipolar depression, dysthymia and seasonal effective disorder). These compounds are also useful for treating ophthalmic disorders. The present invention is also directed to treating additional neurological disorders, such as dystonia (movement disorder), sleep disorder, muscle relaxation and urinary incontinence. In addition, these compounds are useful for memory/cognition enhancement, i.e., treating memory, learning or cognitive deficits. The present invention is also useful in the treatment of HIV infection. Finally, the present invention is directed to the use of these compounds for controlling pain, e.g. as analgesic agents, and the treatment of migraine, acute pain or persistent pain. They can be used prophylactically and also to relieve the symptoms associated with a migraine episode.

The conopeptides, their derivatives and their salts, have anticonvulsant activity in Frings audiogenic seizure susceptible mice and in syndrome-specific seizure animal models. These peptides also have activity in animal pain models. These peptides further have activity in in vitro assays for protection from neurotoxicity. These peptides also have activity in animal models for Parkinson's disease. Thus, the peptides of the present invention are useful as anticonvulsant agents, as neuroprotective agents, as analgesic agents, for managing pain and for treating neurodegenerative disorders. The peptides are administered to patients as described further below.

These peptides are sufficiently small to be chemically synthesized. General chemical syntheses for preparing the foregoing peptides are described in PCT published application WO 98/03189. The peptides are synthesized by a suitable method, such as by exclusively solid-phase techniques, by partial solid-phase techniques, by fragment condensation or by classical solution couplings. The peptides are also synthesized using an automatic synthesizer. Conopeptides of the present invention can also be obtained by isolation and purification from specific Conus species using the technique described in in PCT published application WO 98/03189.

Although the conopeptides of the present invention can be obtained by purification from cone snails, because the amounts of peptide obtainable from individual snails are very small, the desired substantially pure peptides are best practically obtained in commercially valuable amounts by chemical synthesis using solid-phase strategy. For example, the yield from a single cone snail may be about 10 micrograms or less of peptide. By “substantially pure” is meant that the peptide is present in the substantial absence of other biological molecules of the same type; it is preferably present in an amount of at least about 85% purity and preferably at least about 95% purity.

The peptides of the present invention can also be produced by recombinant DNA techniques well known in the art. Such techniques are described by Sambrook et al. (1989). The peptides produced in this manner are isolated, reduced if necessary, and oxidized, if necessary, to form the correct disulfide bonds.

The conopeptides of the present invention have been found to be antagonists of the excitatory amino acid (EAA) receptors, including the ionotropic glutamate (or EAA) receptors (iGluRs, including NMDA receptors, AMPA receptors and KA receptors) and the G-protein coupled glutamate (or EAA) receptors (mGluRs). For example, conopeptide JG001, has been found to be an antagonist of the NMDA receptor subunits and is useful as anticonvulsant agents, as neuroprotective agents, as analgesic agents, for managing pain and for treating neurodegenerative disorders. The conopeptides of the present invention, as well as their derivatives and salts, are particularly useful as such agents for treating neurologic disorders and psychiatric disorders that result from an overstimulation of excitatory amino acid receptors. That is, the invention pertains particularly to disorders in which the pathophysiology involves excessive excitation of nerve cells by excitatory amino acids or agonists of the ionotropic EAA receptors, such as the NMDA receptor(s), AMPA receptor and KA receptor and of the G-protein coupled EAA receptors. Thus, the conopeptides of the present invention are useful for the treatment and alleviation of epilepsy and as general anticonvulsant agents. The use of the conopeptides of the present invention in these conditions includes the administration of a conopeptide in a therapeutically effective amount to patients in need of treatment. The conopeptides of the present invention can be used to treat the seizures, to reduce their effects and to prevent seizures.

The conopeptides of the present invention are also useful to reduce neurotoxic injury associated with conditions of hypoxia, anoxia or ischemia which typically follows stroke, cerebrovascular accident, brain or spinal chord trauma, myocardial infarct, physical trauma, drownings, suffocation, perinatal asphyxia, or hypoglycemic events. To reduce neurotoxic injury, a conopeptide should be administered in a therapeutically effective amount to the patient within 24 hours of the onset of the hypoxic, anoxic or ischemic condition in order for conopeptide to effectively minimize the CNS damage which the patient will experience.

The conopeptides are further useful for the treatment of Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia and neuronal damage associated with uncontrolled seizures. The administration of a conopeptide in a therapeutically effective amount to a patient experiencing such conditions will serve to either prevent the patient from experiencing further neurodegeneration or it will decrease the rate at which neurodegeneration occurs. In addition, the conopeptides can be administered in adjunct with conventional treatment agents to reduce the amount of such agents which need to be used.

The conopeptides of the present invention are also useful for treating chemical toxicity (such as addiction, morphine tolerance, opiate tolerance, opioid tolerance and barbiturate tolerance), anxiety, major depression, manic-depressive illness, obsessive-compulsive disorder, schizophrenia, mood disorders (such as bipolar disorder, unipolar depression, dysthymia and seasonal effective disorder), dystonia (movement disorder), sleep disorder, muscle relaxation, urinary incontinence, HIV infection and ophthalmic indications. In treating these conditions, a therapeutically effective amount of a conopeptide is administered to a patient to completely treat the condition or to ease the effects of the condition. In addition, the conopeptides are useful for memory/cognition enhancement (treating memory, learning or cognitive deficits), in which case a therapeutically effective amount of a conopeptide is administered to enhance memory or cognition.

The conopeptides of the present invention are further useful in controlling pain, e.g., as analgesic agents, and the treatment of migraine, acute pain or persistent pain. They can be used prophylactically or to relieve the symptoms associated with a migraine episode, or to treat acute or persistent pain. For these uses, a conopeptide is administered in a therapeutically effective amount to overcome or to ease the pain.

The anticonvulsant effects of the conopeptide JG001 has been demonstrated in animal models. In rodents, conopeptide JG001 is effective against supramaximal tonic extension seizures produced by maximal electroshock and threshold seizures induced by subcutaneous (s.c.) pentylenetetrazole or picrotoxin. As described in further detail below, conopeptide JG001 was found to have a protective index of 20. Conopeptide JG001 is also effective against focal seizures induced by aluminum hydroxide injection into the pre- and post-central gyri of rhesus monkeys. Conopeptide JG001 , when administered to patients with refractory complex partial seizures, may markedly reduce seizure frequency and severity. Thus, conopeptide JG001 is useful as anticonvulsant agents. Moreover, the clinical utility of conopeptide JG001 as a therapeutic agent for epilepsy may include generalized tonic-clonic and complex partial seizures.

The neuroprotective effects of conopeptide JG001 is demonstrated in laboratory animal models. In these models, conopeptide JG001 protects against hypoxic damage to the hippo-campal slice in vitro. In neonate rats, conopeptide JG001 reduces the size of cortical infarcts and amount of hippocampal necrosis following bilateral carotid ligation and hypoxia. Thus, cono-peptide JG001 are useful as neuroprotective agents. Whereas other anticonvulsants may exhibit neuroprotectant properties (Aldrete et al., 1979; Abiko et al., 1986; Nehlig et al., 1990), these effects often occurred only at high, clinically acheivable doses associated with considerable toxicity (Troupin et al., 1986; Wong et al., 1986). In contrast, conopeptide JG001 exhibits both anticonvulsant and neuroprotectant effects at doses well toerated by animals and humans.

The analgesic or anti-pain activity of conopeptide JG001 is demonstrated in animal models of pain and in animal models of persistent pain. In these models, conopeptide JG001 is (a) effective in nerve injury model studies; (b) effective in reducing the tolerance to opiate analgesics after chronic administration and (c) effective in inhibiting activation of NMDA receptors and thereby inhibiting the release of Substance P by small-diameter, primary, sensory pain fibers. Thus, conopeptide JG001 is useful as analgesic agents and anti-pain agents for the treatment of acute and persistent pain. Conopeptide JG001 is also useful for treating addiction, morphine/opiate/opioid tolerance or barbiturate tolerance.

The anti-neurodegenerative disease or neuroprotective activity of conopeptide JG001 is demonstrated in animal models of Parkinson's disease. Conopeptide JG001 is effective in reversing the behavioral deficits induce by dopamine depletion. Conopeptide JG001 shows behavioral potentiation, especially locomotor activity. Conopeptide JG001 enhances the effect of L-DOPA in reversing the behavioral deficits induce by dopamine depletion. Thus, conopeptide JG001 is effective neuroprotective agent and anti-neurodegenerative disease agent.

The effect of conopeptide JG001 on muscle control is demonstrated in animals. At low doses, conopeptide JG001 is effective in hampering voiding at the level of the urethra. At higher doses, conopeptide JG001 is effective in eliminating all lower urinary tract activity. In the animal studies, it appears that conopeptide JG001 is more discriminatory in their inhibitory effects on striated sphincter than on bladder when compared with other NMDA antagonists. Thus, conopeptide peptide JG001 can be dosed in such a way so as to selectively decrease bladder/sphincter dyssynergia, especially in spinal cord injured patients, and are therefore useful for treating urinary incontinence and muscle relaxation.

In addition to the above medical uses, several of the conopeptides of the present invention have agricultural uses. The conopeptides derived from worm hunting Conus species contain N-terminal sequences distinctive from that of piscivorous species in that residue 2 is invariably aromatic. These peptidic toxins are directed at invertebrate glutamate receptors and therefore have have agricultural applications, e. for the control of nematodes, parasitic worms and other worms.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic. efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:
(a) pump (see, e.g., Luer & Hatton (1993), Zimm et al. (1984) and Ettinger et al. (1978));
(b), microencapsulation (see, e.g., U.S. Pat. Nos. 4,352,883; 4,353,888; and 5,084,350);
(c) continuous release polymer implants (see, e.g., U.S. Pat. No. 4,883,666);
(d) macroencapsulation (see, e.g., U.S. Pat. Nos. 5,284,761, 5,158,881, 4,976,859 and 4,968,733 and published PCT patent applications WO92/19195, WO 95/05452);
(e) naked or unencapsulated cell grafts to the CNS (see, e.g., U.S. Pat. Nos. 5,082,670 and 5,618,531);
(f) injection, either subcutaneously, intravenously, intra-arterially, intramuscularly, or to other suitable site; or
(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in an therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or spealists, and typically takes account of the disorder to be treated, the condition of the individual patient, the site of delivery, the method of administration and other factors known to practitioners. Examples of techniques and protocols can be found in Remington 's Parmaceutical Sciences.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

For the treatment of pain, if the route of administration is directly to the CNS, the dosage contemplated is from about 1 ng to about 100 mg per day, preferably from about 100 ng to about 10 mg per day, more preferably from about 1 μg to about 100 μg per day. If administered peripherally, the dosage contemplated is somewhat higher, from about 100 ng to about 1000 mg per day, preferably from about 10 μg to about 100 mg per day, more preferably from about 100 pg to about 10 mg per day. If the conopeptide is delivered by continuous infusion (e.g., by pump delivery, biodegradable polymer delivery or cell-based delivery), then a lower dosage is contemplated than for bolus delivery.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Ausubel et al., 1992; Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is described by reference to the following Examples, which are offered by way of illustration and are not intended to limit the invention in any manner. Standard techniques well known in the art or the techniques specifically described below were utilized.

### Example 1

### Isolation of DNA Encoding Conopeptide JG001

DNA coding for conopeptide JG001 (Gly-Xaa₁-Asp-Xaa₁-Val-Ser-Gln-Met-Ser-Xaa₂-Xaa₁-Ile-Leu-Arg-Xaa₁-Leu-Glu-Leu-Gln-Xaa₂; Xaa₁ and Xaa₂ are as X₁ and X₂ above; SEQ ID NO:33); was isolated and cloned in accordance with conventional techniques. The DNA was isolated by reverse transcription-PCR using Conus aurisiacus venom duct mRNA and primer CCon8 as the forward primer and the primer LibU as the reverse primer. The sequences for these primers are as follows:

| | |
|---|---|
| CCon8: | |
| CAGGATCCTGTATCTGCTGGTGCCCCTGGTG | (SEQ ID NO: 34) |
| and | |
| LibU: | |
| AAGCTCGAGTAACAACGCAGAGT. | (SEQ ID NO: 35) |

### Example 2

### In vivo Activity of Conopeptide JG001 in Frings Audiogenic Seizure Susceptible Mice

In vivo anticonvulsant activity of conopeptide JG001 (in which Xaa₁ and Xaa₂ are each Gla) was analyzed in Frings audiogenic seizure susceptible mice as described by White et al. (1992). The results for conopeptide JG001 are shown in Tables 1-3.

TABLE 1
Effect of Conopeptide JG001 on the Audiogenic Seizure
Susceptibility of Frings Mice Following i.c.v. Administration

| | | |
|---|---|---|
| Dose | # Protected/# Tested | # Toxic/# Tested |

| (pmol, i.c.v.) | 30 min. | 120 min. | 30 min. | 120 min. |
|---|---|---|---|---|
| 300 | 4/4 | 4/4 | 0/4 | 0/4 |
| 1000 | 3/4 | 4/4 | 2/4 | 1/4 |

TABLE 2
Time Effect of Conopeptide JG001 Against Audiogenic
Seizure Susceptibility of Frings Mice Following i.c.v. Administration

| | | Time (hrs) | | | | | |
|---|---|---|---|---|---|---|---|
| | Dose | ¼ | ½ | 1 | 2 | 4 | Reference |
| # Prot./# Tested | 75 pmol | — | 4/4 | — | 3/4 | — | HA2: 143 |
| # Toxic/# Tested | 75 pmol | — | 0/4 | — | 0/4 | — | HA2: 143 |

TABLE 3
Effect of Conopeptide JG001 on the Audiogenic Seizure
Susceptibility of Frings Mice Following i.c.v. Administration

| | | # | | | |
|---|---|---|---|---|---|
| | | Protected/ | | # Toxic/ | |
| | Seizure | # Tested | | # Tested | |
| Dose | Score ± | (at 30 | ED₅₀ | (at 30 | TD₅₀ |
| (pmol) | S.E.M. | min) | (pmol) | min) | (pmol) |
| 18.75 | 5 ± 0 | 0/8 | | | |
| 37.5 | 3.25 ± 0.86 | 3/8 | | | |
| 56.25 | 2.5 ± 0.95 | 4/8 | 46.79 | | |
| 75 | 0.13 ± 0.13 | 8/8 | (33.82-58.33)* | | |
| 300 | | | | 1/8 | |
| 1000 | | | | 3/8 | |

*95% confidence interval
Ref: HA2: 142-145

Conopeptide JG001 yielded an effective dose (ED₅₀) of 46.79 pmol, with a 95% confidence interval of 33.82-58.33 pmol. Furthermore, conopeptide JG001 yielded a toxic dose (TD₅₀) of 1000 pmol (toxicity to ⅜ animals). The dose required to elicit neurotoxicity was >20 times greater than the effective dose (TD₅₀/ED₅₀=1000/46.79=21.37=Protective Index, PI). The therapeutic dose of typical anti-seizure medications is close to the toxic dose (typical PI=2-3). Since the protective index is high for conopeptide JG001 , this peptide will be better tolerated than previous anti-convulsant agents.

### Example 3

### In vivo Activity of Conopeptide JG001 in CF No. 1 Mice

In vivo anticonvulsant activity of conopeptide JG001 is analyzed in CF No. 1 mice as described by White et al. (1995), using the maximal electroshock, subcutaneous pentylenetetrazole (Metrazol) seizure threshold and threshold tonic extension test. Conopeptide JG001 is found to have anticonvulsant activity.

### Example 4

### In Vivo Activity of Conopeptide JG001 in Pentylenetetrazole-Induced Threshold Seizure Model

The in vivo activity of conopeptide JG001 is analyzed using timed intravenous infusion of pentylenetetrazole (White et al., 1995). At time to peak effect, the convulsant solution (0.5% pentylenetetrazole in 0.9% saline containing 10 U.S.P. units/ml heparin sodium) is infused into the tail vein at a constant rate of 0.34 ml/min. The time in seconds from the start of the infusion to the appearance of the first twitch and the onset of clonus is recorded for each drug treated or control animal. The times to each endpoint are converted to mg/kg of pentylenetetrazole for each mouse, and mean and standard error of the mean are calculated. It is found that conopeptide JG001 elevates the i.v. pentylenetetrazole seizure threshold.

### Example 5

### In Vivo Activity of Conopeptide JG001 in Parkinson's Disease Animal Model

The anti-Parkinsonian potential of conopeptide JG001 is examined in rats with unilateral lesions of the nigrostriatal dopamine system. The unilateral lesions are created by local infusion of the neurotoxin 6-hydroxydopamine (6-OHDA) into the right substantia nigra of anesthetized rats. The rats recovered for two weeks at which time they are anesthetized and guide cannulae implanted into the brain, ending in the right lateral ventricle. The guide cannulae are kept patent with a stylet placed in the guide cannula. One week later, the rats are placed in a cylindrical Plexiglas®) cage, the stylet is removed, and an infusion cannula is inserted into the guide. The infusion cannula is attached to a syringe on an infusion pump which delivered conopeptide JG001 (0.5 mM, 5.0 mM or 50 mM) or control vehicle at a rate of 1 μl/min for a total injection of 2 μl (1 nmol/2 μl). Fifteen minutes after the injection of conopeptide JG001, L-Dopa (4 mg/kg ip) is injected. The number of full rotations contralateral and ipsilateral to the dopamine-depleted hemisphere is then counted for 2 minutes, every 10 minutes, for 2 hours. A video of the rats is also made to follow the behavioral potentiation of the treatment. It is seen that the tested compound reverses the behavioral deficits induced by dopamine depletion. In addition to the above tests, the in vivo activity of conopeptide JG001 in combination with SKF 38393 is compared with that of SKF 38393 alone. It is seen that the combination of conopeptide JG001 and SKF 38393 demonstrates increased activity.

### Example 6

### In vivo Activity of Conopeptide JG001 in Pain Models

The anti-pain activity of conopeptide JG001 is shown in several animal models. These models include the nerve injury model (Chaplan, et al., 1997), the nocioceptive response to s.c. formalin injection in rats (Codene, 1993) and an NMDA-induced persistent pain model (Liu, et al., 1997). In each of these models it is seen that the conopeptides and conopeptide derivatives have analgesic properties.

More specifically, this study evaluates the effect of intrathecal administration of conopeptide JG001 in mice models of nocioceptive and neuropathic pain. For nocioceptive pain, the effect of the conopeptide JG001 is studied in two different tests of inflammatory pain. The first is the formalin test, ideal because it produces a relatively short-lived, but reliable pain behavior that is readily quantified. There are two phases of pain behavior, the second of which is presumed to result largely from formalin- evoked inflammation of the hind paw. Conopeptide JG001 is administered 10 minutes prior to injection of formalin. The number of flinches and/or the duration of licking produced by the injection is monitored. Since the first phase is presumed to be due to direct activation of primary afferents, and thus less dependent on long term changes in the spinal cord, conopeptide JG001 is presumed to have greatest effect on the magnitude of pain behavior in the second phase.

The mechanical and thermal thresholds in animals that received an injection of complete Freund's adjuvant into the hind paw are also studied. This produces a localized inflammation including swelling of the hind paw and a profound decrease in mechanical and thermal thresholds, that are detected within 24 hours after injection. The changes in thresholds in rats that receive conopeptide JG001 are compared with those of rats that receive vehicle intrathecal injections.

To evaluate the contribution of long term, NMDA receptor-mediated changes to neuropathic (i.e., nerve injury-induced) behavior, a modification of the Seltzer model of pain that has been adapted for the mouse is used. A partial transection of the sciatic nerve is first made. This also produces a significant drop in mechanical and thermal thresholds of the partially denervated hind paw. In general, the mechanical changes are more profound. They peak around 3 days after surgery and persist for months.

An important issue is whether the drugs are effective when administered after the pain model has been established, or whether they are effective only if used as a pretreatment. Clearly, the clinical need is for drugs that are effective after the pain has developed. To address this issue, animals are studied in which conopeptide JG001 is administered repeatedly, after the inflammation (CFA) or nerve injury has been established. In these experiments, conopeptide JG001 is injected daily by the intrathecal (i.t.) route. The mechanical and thermal thresholds (measured, respectively, with von Frey hairs in freely moving animals and with the Hargreave's test, also in freely moving animals) are repeated for a 2 to 4 week period after the injury is induced and the changes in pain measured monitored over time.

### Example 7

### Isolation of DNA Encoding Conopeptides

DNA coding for conopeptides was isolated and cloned in accordance with conventional techniques using general procedures well known in the art, such as described in Example 1 or in Olivera et al. (1996). Alternatively, cDNA libraries was prepared from Conus venom duct using conventional techniques. DNA from single clones was amplified by conventional techniques using primers which correspond approximately to the M13 universal priming site and the M13 reverse universal priming site. Clones having a size of approximately 300-500 nucleotides were sequenced and screened for similarity in sequence to known conopeptides similar to conopeptide JG001 isolated in Example 1. The DNA sequences, encoded propeptide sequences and sequences of the mature toxins are set forth in Table 4. DNA sequences coding for the mature toxin can also be prepared on the basis of the DNA sequences set forth on these pages. An alignment of the conopeptides of the present invention with respect to conantokin G is set forth in Table 5. An alignment of the peptides of the present invention is set forth in Table 6.

TABLE 4

| | |
|---|---|
| Name: | Conotoxin-C1 |
| Species: | catus |
| Cloned: | Yes |

DNA Sequence:
GCGATGCAACTGTACACGTATCTGTATCTGCTGGTGGCGCTGGTGACCTTCCACCTAATCCTAGGCACGG
GCACAGTAGATCATGGAGGCGCACTGACTGAAGGGCGTTCGGGTGACGCCACAGCGGTGAGACCTGAGCC
TGTCCTCCTGCAGAAATCCGCTGCCCGCAGCACCGAGGACAGTGGCAAGGACAGGTTGACTCAGATGAAG
AGGATTCTCAAAAAGCAAGGAAACACGGCTAAAAGCGAGGAAGAGCTACTACGAGAGGATGTAGAGACTG
TTTTAGAACTCGAAAGGAATGGAAAAAGATAATCAAGCTGAGTGTTCCACGTGACACTCGTCAGTTCTAA
AGTCCCCAGATAAATCGTTCCCTATTTTGCCACATTCTTTCTTTCTCTTTTCATTTAATTCCCCAAATCT
TTCATGTTTATT (SEQ ID NO:36)
Translation:
MQLYTYLYLLVPLVTFHLILGTGTLDHGGALTERRSGDATALRPEPVLLQKSAARSTDDSGKDRLTQMKR
ILKKQGNTAKSDEELLREDVETVLELERNGKR (SEQ ID NO:37)
Toxin Sequence:
Ser-Asp-Xaa1-Xaa1-Leu-Leu-Arg-Xaa1-Asp-Val-Xaa1-Thr-Val-Leu-Xaa1-Leu-
Xaa1-Arg-Asn-# (SEQ ID NO:38)

| | |
|---|---|
| Name: | Conotoxin-C2 |
| Species: | catus |
| Cloned: | Yes |

DNA Sequence:
GCGATGCAACTGTACACGTATCTGTATCTGCTGGTGCCCCTGGTGACCTTCCACCTAATCCTAGGCACGG
GCACACTAGATCATGGAGGCGCACTGACTGAACGCCGTTCGGGTGACGCCACAGGGCTGAGACCTGAGCC
TGTCCTCCTGCAGAAATCCGCTGCCCGCAGCACCGACGACAGTGGCAAGGACAGGTTGACTCAGATGAAG
AGGATTCTCAAAAAGCAAGGAAACACGGCTAAAGGCGACGAAGAGCTACTACGAGAGGATGTAGAGACTG
TTTTAGAACTCGAAAGGGATGGAAAAAGATAATCAAGCTGAGTGTTCCACGTGGCACTCGTCAGTTCTAA
AGTCCCCAGATAAATCGTTCCCTATTTTGCCACATTCTTTCTTTCTCTTTTCATTTAATTCCCCAAATCT
TTCATGTTTATT (SEQ ID NO:39)
Translation:
MQLYTYLYLLVPLVTFHLILGTGTLDHGGALTERRSGDATALRPEPVLLQKSAARSTDDSGKDRLTQMKR
ILKKQGNTAKGDEELLREDVETVLELERDGKR (SEQ ID NO:40)
Toxin Sequence:
Gly-Asp-Xaa1-Xaa1-Leu-Leu-Arg-Xaa1-Asp-Val-Xaa1-Thr-Val-Leu-Xaa1-Leu-
Xaa1-Arg-Asp-# (SEQ ID NO:41)

| | |
|---|---|
| Name: | Conotoxin-C3 |
| Species: | catus |
| Cloned: | Yes |

DNA Sequence:
GCGATGCAACTGTACACGTATCTGTATCTGCTGGCGCCCCTGGTGACCTTCCACCTAATCCTAGGCACGG
GCACACTAGATCATGGAGGCGCACTGACTGAACGCCGTTCGGGTGACGCCACAGCGCTGAGACCTGAGCC
TGTCCTCCTGCAGAAATCCGCTGCCCGCAGCACCGACGACAGTGGCAAGGACAGGTTGACTCAGATGAAG
AGGATTCTCAAAAAGCAAGGAAACACGGCTAAAAGCGACGAAGAGCTACTACGAGAGGATGTAGAGACTG
TTTTAGAACCCGAAAGGAATGGAAAAAGATAATCAAGCTGAGTGTTCCACGTGACACTCGTCAGTTCTAA
AGTCCCCAGATAAATCGTTCCCTATTTTGCCACATTCTTTCTTTCTCTTTTCATTTAATTCCCCAAATCT
TTCATGTTTATT (SEQ ID NO:42)
Translation:
MQLYTYLYLLAPLVTFHLILGTGTLDHGGALTERRSGDATALRPEPVLLQKSAARSTDDSGKDRLTQMKR
ILKKQGNTAKSDEELLREDVETVLEPERNGKR (SEQ ID NO:43)
Toxin Sequence:
Ser-Asp-Xaa1-Xaa1-Leu-Leu-Arg-Xaa1-Asp-Val-Xaa1-Thr-Val-Leu-Xaa1-Xaa3-
Xaa1-Arg-Asn-# (SEQ ID NO:44)

| | |
|---|---|
| Name: | Conotoxin-C4 |
| Species: | catus |
| Cloned: | Yes |

DNA Sequence:
GCGATGCAACTGtACACGTATCTGTATCTGCTGGTGTCCCTGGTGACCTTCCACCTAATCCTAGGCACGG
GCACACTAGATCATGGAGGCGCACTGACTGAACGCCGTTTGGCTGACGCCACAGCGCTGGAAGCTGAGCC
TGTCCTCCTGCAGAAATCCGCTGCCCGCAGCACCGACAACAATGGCAAGGACAGGTCGACTCAGATGAGG
AGGATTCTCAAAAAGCAAGGAAACACGGCTAGAATCGAGGAAGGTCTGATAGAGGATCTGGAGACCGCTA
GAGAACGCGACAGTGGAAAAAGATAATCAAGCTGAGTGTTCCACGTGACACTCATCAGTTCTAAAGTCCC
CAGATAAATCGTTCCCTATTTTTGCCACATTCTTTCTTCCTCTTTTCGTTTAATTCCCCAAATCTTTCAT
GTTTATT (SEQ ID NO:45)
Translation:
MQLYTYLYLLVSLVTFHLILGTGTLDHGGALTERRLADATALEAEPVLLQKSAARSTDNNGKDRSTQMRR
ILKKQGNTARIEEGLIEDLETARERDSGKR (SEQ ID NO:46)
Toxin Sequence:
Ile-Xaa1-Xaa1-Gly-Leu-Ile-Xaa1-Asp-Leu-Xaa1-Thr-Ala-Arg-Xaa1-Arg-Asp-
Ser-# (SEQ ID NO:47)

| | |
|---|---|
| Name: | Conotoxin-C5 |
| Species: | catus |
| Cloned: | Yes |

DNA Sequence:
GCGATGCAACTGTACACGTATCTGTATCTGCTGGTGTCCCTGGTGACCTTCCACCTAATCCTAGGCACGG
GCACACTAGATCATGGAGGCGCACTGACTGAACGCCGTTTGGCTGACGCCACAGCGCTGGAAGCTGAGCC
TGTCCTCCTGCAGAAATCCGCTGCCCGCAGCACCGACAACAATGGCAAGGACAGGTCGACTCAGATGAGG
AGGATTCTCAAAAAGCAAGGAAACACGGCTAGAATCGAGGAAGGTCTGATAGAGGATCTGGAGGCTGCTA
GAGAACGCGACAGTGGAAAAAGATAATCAAGCTGAGTGTTCCACGTGACACTCATCAGTTCTAAAGTCCC
CAGATAAATCGTTCCCTATTTTTGCCACATTCTTTCTTCCTCTTTTCGTTTAATTCCCCAAATCTTTCAT
GTTTATT (SEQ ID NO:48)
Translation:
MQLYTYLYLLVSLVTFHLILGTGTLDHGGALTERRLADATALEAEPVLLQKSAARSTDNNGKDRSTQMRR
ILKKQGNTARIEEGLIEDLEAARERDSGKR (SEQ ID NO:49)
Toxin Sequence:
Ile-Xaa1-Xaa1-Gly-Leu-Ile-Xaa1-Asp-Leu-Xaa1-Ala-Ala-Arg-Xaa1-Arg-Asp-
Ser-# (SEQ ID NO:50)

| | |
|---|---|
| Name: | Conotoxin-C6 |
| Species: | catus |
| Cloned: | Yes |

DNA Sequence:
GCGATGCAACTGTACACGTATCTGTATCTGCTGGTGCCCCTGGTGACCTTCCACCTAATCCTAGGCACGG
GCACACTAGATCATGGAGGCGCACTGACTGAACGCCGTTCGGCTGACGCCACAGCGCTGAAACCTGAGCC
TGTCCTCCTGCAGAAATCCGGTGCCCGCAGCACCGACGACAATGGCAAAGACAGGTTGACTCACATGAAG
AGGATTCTCAAAAAACGAGCAAACAAAGCCAGAGGCGAACCAGAAGTTGGAAGCATACCGGAGGCAGTAA
GACAACAAGAATGTATAAGAAATAATAATAATCGACCTTGGTGTCCCAAGTGACACTCGTCAGTTCTAAA
GTCTCCAGATAGATCGTTCCCTATTTTTGCCACACTCTTTCTTTCTCTTTTCATTTAAGTTCCCCAAATC
TTTCATGTTTATT (SEQ ID NO:51)
Translation:
MQLYTYLYLLVPLVTFHLILGTGTLDHGGALTERRSADATALKPEPVLLQKSAARSTDDNGKDRLTHMKR
ILKKRANKARGEPEVGSIPEAVRQQECIRNNNNRPWCPK (SEQ ID NO:52)
Toxin Sequence:
Gly-Xaa1-Xaa3-Xaa1-Val-Gly-Ser-Ile-Xaa3-Xaa1-Ala-Val-Arg-Gln-Gln-Xaa1-
Cys-Ile-Arg-Asn-Asn-Asn-Asn-Arg-Xaa3-Xaa4-Cys-Xaa3-Lys-\hat{ } (SEQ ID NO:
53)

| | |
|---|---|
| Name: | Conotoxin-Bu1 |
| Species: | bullatus |
| Cloned: | Yes |

DNA Sequence:
GCGATGCAACTGTACACGTATCTGTATCTGCTGGTGCCCTTGGTGACCTTCCACCTAATCCTGGGCACGG
GCACACTAGATCATGGAGGCGCACTGACTGAACGCCGTTCGGCTGACGCCACAGCACTGAAACCTGAGCC
TGTCCTCCTGCAGAAAACCGCTGCCCGCAGCACCGACGACAATGGCAAGAAGAGGCTGACTCAGAGGAAG
AGGATTCTCAAAAAGCGAGGAAACACGGCTAGAAACCCCGAAACTTATATAGAGATTGTGGAGATTTCTA
GGGAACTCGAAGAGATTGGAAAAAGATAATCAAGCTGGGTGTTCCACGTGACACTCGTCAGTTCTGAAGT
CCCGAGGTAGATCGTTCCCTATTTTTGCCACACTCTTTCTTTCTCTTTTCATTTAATTCCCCAAATCTTT
CATGTTTATT (SEQ ID NO:54)
Translation:
MQLYTYLYLLVPLVTFHLILGTGTLDHGGALTERRSADATALKPEPVLLQKTAARSTDDNGKKRLTQRKR
ILKKRGNTARNPETYIEIVEISRELEEIGKR (SEQ ID NO:55)
Toxin Sequence:
Asn-Xaa3-Xaa1-Thr-Xaa5-Ile-Xaa1-Ile-Val-Xaa1-Ile-Ser-Arg-Xaa1-Leu-
Xaa1-Xaa1-Ile-# (SEQ ID NO:56)

| | |
|---|---|
| Name: | Conotoxin-Bu2 |
| Species: | bullatus |
| Cloned: | Yes |

DNA Sequence:
GCGATGCAACTGTACACGTATCTGTATTTGCTGGTGCCCTTGGTGACCTTCCACCTAATCCTGGGCACGG
GCACACTAGATCATGGAGGCGCACTGACTGAACGCCGTTCGGCTGACGCCACAGCGCTGAAACCTGAGCC
TGTCCTCCTGCAGAAAACCGCTGCCCGCAGCACCGACGACAATGGCAAGAAGAGGCTGACTCAGAGGAAG
AGGATTCTCAAAAAGCGAGGAAACACGGCTAGAAACCCCGAAACTTATTATAATTTAGAGCTTGTGGAGA
TTTCTAGGGAACTCGAAGAAATTGGAAAAAGATAATCAAGCTGGGTGTTCCACGTGACACTCGTCAGTTC
TTAAGTCCCGAGGTAGATCGTTCCCTATTTTTGCCACACTCTTTCTTTCTCTTTTCATTTAATTCCCCAA
ACTTTCATGTTTATT (SEQ ID NO:57)
Translation:
MQLYTYLYLLVPLVTFHLILGTGTLDHGGALTERRSADATALKPEPVLLQKTAARSTDDNGKKRLTQRKR
ILKKRGNTARNPETYYNLELVEISRELEEIGKR (SEQ ID NO:58)
Toxin Sequence:
Asn-Xaa3-Xaa1-Thr-Xaa5-Xaa5-Asn-Leu-Xaa1-Leu-Val-Xaa1-Ile-Ser-Arg-
Xaa1-Leu-Xaa1-Xaa1-Ile-# (SEQ ID NO:59)

| | |
|---|---|
| Name: | Conotoxin-Bt1 |
| Species: | betulinus |
| Cloned: | Yes |

DNA Sequence:
GCGATGCAACTGTACACGTATCTGTATCTGCTGGTGCCCCTGGTGACCTTCTACCTAATCCTAGGCACGG
GCACGCTAGGTCATGGAGGCGCACTGACTGAACGCCGTTTGGCTGATGCCACAGCGCTGAAACCTGAGCC
TGTCCTCCTGCAGAAATCCGCCGCCCGCAGCACCGACGACAATGGCAAGGACAGGTTGACTCAGATGATC
AGGATTCTCAAAAAGCGAGGAAACATGGCCAGAGGCGGCGAAGAAGTTAGAGAGTCTGCAGAGACTCTTC
ATGAACTGACGCCGTAGGAAAAAGAAAAAGATTAATCAAGCTGGGTGTCCCACGTGACACTCGTCAGTTC
TAAAGTCCCCAGTTTCCTATCTTTGCCACGTTTCTTTTTCTTTTCATTCAATTCCCCAAATCTTTCATGT
TTATT (SEQ ID NO:60)
Translation:
MQLYTYLYLLVPLVTFYLILGTGTLGHGGALTERRLADATALKPEPVLLQKSAARSTDDNGKDRLTQMIR
ILKKRGNMARGGEEVRESAETLHELTP (SEQ ID NO:61)
Toxin Sequence:
Gly-Gly-Xaa1-Xaa1-Val-Arg-Xaa1-Ser-Ala-Xaa1-Thr-Leu-His-Xaa1-Leu-Thr-
Xaa3-\hat{ } (SEQ ID NO:62)

| | |
|---|---|
| Name: | Conotoxin-Bt2 |
| Species: | betulinus |
| Cloned: | Yes |

DNA Sequence:
GCGATGCAACTGTATACGTATCTGTATCTGCTGGTGCCGCTGGTGACCTTCTACCTAATCCTAGGCACGG
GCACGCTAGGTCATGGAGGCGCACTGACTGAACGCCGTTTGGCTGACGCCACAGCGCTGAAACCTGAGCC
TGTCCTCCTGCAGAAATCCGCCGCCCGCAGCACTGACGACAATGGCAAGGACAGGTTGACTCAGATGATC
AGGATTCTCAAAAAGCGAGGAAACATGGCCAGAGGCGGCGAAGAAGTTAGAGAGTCTGCAGAGACTCTTC
ATGAAATCACGCCGTAGGAAAAAGAAAAAGATTAATCAAGCTGGGTGTTCCACGTGACACTCGCCAGTTC
TAAAGTCCCCAGTTTCCTATCTTTGCCAGGTTTCTTTCTCTTTTCATTCAATTCCCCAAATCTTTCATGT
TTATT (SEQ ID NO:63)
Translation:
MQLYTYLYLLVPLVTFYLILGTGTLGHGGALTERRLADATALKPEPVLLQKSAARSTDDNGKDRLTQMIR
ILKKRGNMARGGEEVRESAETLHEITP (SEQ ID NO:64)
Toxin Sequence:
Gly-Gly-Xaa1-Xaa1-Val-Arg-Xaa1-Ser-Ala-Xaa1-Thr-Leu-His-Xaa1-Ile-Thr-
Xaa3-\hat{ } (SEQ ID NO:65)

| | |
|---|---|
| Name: | Conotoxin-Bt3 |
| Species: | betulinus |
| Cloned: | Yes |

DNA Sequence:
GCGATGCAACTGTACACGTATCTGTATCTGCTGGTGCCCCTGGTGACCTTCTACCTAATCCTAGGCACGG
GCACGCTAGGTCATGGAGGCGCACTGACTGAACGCCGTTTGGCTGACGCCACAGCGCTGAAACCTAAGCC
TATCCTCCTGCAGAAATCCGCCGCCCGCAGCACTGACGACAATGGCAAGGACAGGTTGACTCAGATGATC
AGGATTCTCAAAAAGCGAGGAAACATGGGCAGAGACGGCGAAGAAGTCAGAGAGGCTGCAGAGACTCTTA
ATGAACTCACGCCGTAGGAAAAAGAAAAAGATTAATCAAGCTGGGTGTTCCACGTGACACTCGTCAGTTC
TAAAGTACCCAGTTTCCTATCTTTGCCACGTTTCTTTTTCTTTCCATTCAATTCCCCAAATCTTTCATGT
TTATT (SEQ ID NO:66)
Translation:
MQLYTYLYLLVPLVTFYLILGTGTLGHGGALTERRLADATALKPKPILLQKSAARSTDDNGKDRLTQMIR
ILKKRGNMGRDGEEVREAAETLNELTP (SEQ ID NO:67)
Toxin Sequence:
Asp-Gly-Xaa1-Xaa1-Val-Arg-Xaa1-Ala-Ala-Xaa1-Thr-Leu-Asn-Xaa1-Leu-Thr-
Xaa3-\hat{ } (SEQ ID NO:68)

| | |
|---|---|
| Name: | Conotoxin-Bt4 |
| Species: | betulinus |
| Cloned: | Yes |

DNA Sequence:
GCGATGCAACTGTACACGTATCTGTATCTGCTGGTGCCCCTGGTGACCTTCCACCTAATCCTAGGCACGG
GCACGCTAGGTCATGGAGGCGGACTGACTGAAAGCCGTTCGGCTGACGCCACAGCACTGAAACCAGGGCC
TGTCCTCCTGCAGAAATCCGCTGCCCGCAGCACCGACGACAATGGCAAGGACAGGTTGACTCAGATGAAG
AGGACTCTCAAAAAGCGAGGAAACACGGCCAGAGGCTACGAAGATGATAGAGAGATTGCAGAGACTGTTA
GAGAACTCGAGGAAGCAGGAAAATGAAAAAGATTAATCAAGCTGGGTGTTCCACGTGACACTTGTCAGTT
CTAAAGTCCCCAGATAGATCGTTCCCTATTTTTGCCACATTCTTTTTTTCTCTTTTCATTTAATTCCCCA
AATCTTTCATGTTTATT (SEQ ID NO:69)
Translation:
MQLYTYLYLLVPLVTFHLILGTGTLGHGGALTESRSADATALKPGPVLLQKSAARSTDDNGKDRLTQMKR
TLKKRGNTARGYEDDREIAETVRELEEAGK (SEQ ID NO:70)
Toxin Sequence:
Gly-Xaa5-Xaa1-Asp-Asp-Arg-Xaa1-Ile-Ala-Xaa1-Thr-Val-Arg-Xaa1-Leu-Xaa1-
Xaa1-Ala-# (SEQ ID NO:71)

| | |
|---|---|
| Name: | Conotoxin-Bt5 |
| Species: | betulinus |
| Cloned: | Yes |

DNA Sequence:
GCGATGCAACTGTACACGTATCTGTATCTGCTGGTGCCGCTGGTGACCTTCTACCTAATCCTAGGCACGG
GCACGCTAGGTCATGGAGGCGCACTGACTGAACGCCGTTTGGCTGACGCCACAGCGCTGAAACCTGAGCC
TGTCCTCCTGCAGAAATCCGCCGCCCGCAGCACTGACGACAATGGCAAGGACAGGTTGACTCAGATGATC
AGGATTCTCAAAAAGCGAGGAAACATGGCCAGAGGCGGCGGAGAAGTTAGAGAGTCTGCAGAGACTCTTC
ATGAAATCACGCCGTAGGAAAAAGAAAAAGATTAATCAAGCTGGGTGTTCCACGTGACACTCGTCAGTTC
TAAAGTCCCCAGTTTCCTATCTTTGCCAGGTTTCTTTCTCTTTTCATTCAATTCCCCAAATCTTTCATGT
TTATT (SEQ ID NO:72)
Translation:
MQLYTYLYLLVPLVTFYLILGTGTLGHGGALTERRLADATALKPEPVLLQKSAARSTDDNGKDRLTQMIR
ILKKRGNMARGGGEVRESAETLHEITP (SEQ ID NO:73)
Toxin Sequence:
Gly-Gly-Gly-Xaa1-Val-Arg-Xaa1-Ser-Ala-Xaa1-Thr-Leu-His-Xaa1-Ile-Thr-
Xaa3-\hat{ } (SEQ ID NO:74)

| | |
|---|---|
| Name: | Conotoxin-Af6 |
| Species: | ammiralis |
| Cloned: | Yes |

DNA Sequence:
GCGATGCAACTGTACACGTATCTGTGTCTGCTGGTGCCCCTGGTGACCTTCTACCTAATTCTAGGCACGG
GCACACTAGCTCATGGAGGCGCACTGACCGAACGCCGTTTGGCTCACGCCAGAGTAATAGAACCTGATCC
TGCCCCCCTGGAGAACTCCGCTCTCCGCAGCATCCGACGACAACGACAAGGACAGGATGACTCAGAGGAA
GAGGATTCTCAAAAAGTGATGAAACACGGCCAGAGGCGCGAAAGAAGATAGAAATAATGCGGAGGCTGTT
AGAGAAAGACTCGAAGAAATAGGAAAAAGGTAATCAAGCTGGGTGTTTCACGTGACACTCATCAGTTCTA
AAGTCCCCAGATAGATCGTTCCCTATTTTTGCCATATTCTTTCCTTCTCTTTTCATGTAATTCCCCAAAT
CTTTCATGTTTATT (SEQ ID NO:75)
Translation:
MQLYTYLCLLVPLVTFYLILGTGTLAHGGALTERRLAHARVIEPDPAPLENSALRSIRRQRQGQDDSEEE
DSQKVMKHGQRRERR (SEQ ID NO:76)
Toxin Sequence:
Xaa2-Gly-Gln-Asp-Asp-Ser-Xaa1-Xaa1-Xaa1-Asp-Ser-Gln-Lys-Val-Met-Lys-
His-Gly-Gln-Arg-Arg-Xaa1-Arg-Arg-\hat{ } (SEQ ID NO:77)

| | |
|---|---|
| Name: | Conotoxin-Ep1 |
| Species: | episcopatus |
| Cloned: | Yes |

DNA Sequence:
GCGATGCAACTGTACACGTATCTGTGTCTGCTGGTGCCCCTGGTGACCTTCTACCTAATTCTAGGCACGG
GCACACTAGCTCATGGAGGCGCACTGACTGAACATCGTTCGGCCGACGCCACAGCACTGAAACCTGAGCC
TGTCCTCCTGCAGAAATCCGCTGCCCGCAGCACCGACGACAACGGCAAGGACAGGTTGACTCGGTGGAAG
GGGATTCTCAAAAAGCGAGGAAACACGGCCAGAGGCGGGAAAGATATTGTGGAGACTATTACAGAACTCG
AAAAAATAGGAAAAAGGTAATCAAGCTGGGTGTTCCACGTGACACTCATCAGTTCTAAAGTCCCCAGATA
GATCGTTCCCTATTTTTGCCATATTCTTTCTTTCTCTTTTCATGTAATTCCCCAAATCTTTCATGTTTAT
T (SEQ ID NO:78)
Translation:
MQLYTYLCLLVPLVTFYLILGTGTLAHGGALTEHRSADATALKPEPVLLQKSAARSTDDNGKDRLTRWKG
ILKKRGNTARGGKDIVETITELEKIGKR (SEQ ID NO:79)
Toxin Sequence:
Gly-Gly-Lys-Asp-Ile-Val-Xaa1-Thr-Ile-Thr-Xaa1-Leu-Xaa1-Lys-Ile-#
(SEQ ID NO:80)

| | |
|---|---|
| Name: | Conotoxin-L1 |
| Species: | lynceus |
| Cloned: | Yes |

DNA Sequence:
GCGATGCAACTGTACACGTATCTGTATCTGCTGGTGCCCCTGGTGACCTTCCACCTAATCCTAGGCACGG
GCACACTAGATCATGGAGGCGCACTGACTGAACGCCGTTCGACTGATGCCATAGCACTGAAACCTGAGCC
TGTCCTCCTGCAGAAATCCTCTGCCCGCAGCACCGACGATAATGGCAACGACAGGTTGACTCAGATGAAG
AGGATCCTCAAAAAGCGAGGAAACAAAGCCAGAGGCGAAGAAGAAGTTGCAAAAATGGCGGCAGAGATTG
CCAGAGAAAACGCTGCAAATGGGAAATGATAATCAAGTTGGGTGTTCCACGTGACACTCGTCAGTTCTAA
AGTCCCCAGATAGATCGTTCCCTATTTTTGCCACATTCTTTCTTTCTCTTTTCATTTAATTCCCCAAATC
TTTCATGTTTATT (SEQ ID NO:81)
Translation:
MQLYTYLYLLVPLVTFHLILGTGTLDHGGALTERRSTDAIALKPEPVLLQKSSARSTDDNGNDRLTQMKR
ILKKRGNKARGEEEVAKMAAEIARENAANGK (SEQ ID NO:82)
Toxin Sequence:
Gly-Xaa1-Xaa1-Xaa1-Val-Ala-Lys-Met-Ala-Ala-Xaa1-Ile-Ala-Arg-Xaa1-Asn-
Ala-Ala-Asn-# (SEQ ID NO:83)

| | |
|---|---|
| Name: | Conotoxin-L2 |
| Species: | lynceus |
| Cloned: | Yes |

DNA Sequence:
GCGATGCAACTGTACACGTATCTGTATCTGCTGGTGCCCCTGGTGATCTTCTACCTAATCCTAGGCACGG
GCACGCTAGGTCATGGAGGCACACTGACTGAACGCCGTTCGGCTGATGCCACAGCACTGAAACCTGAGCC
TGTCCTCCTGCAGAAATCCGCTGCCCGCAGCACCGGCGACGATGCCAAGGAGAGGTTGACTCAGACGAAG
AGGATTCGCAAAAAGCGAGCAAACACGACCAGAGGCAAAGAAGAGGATAGAGAGATTGTGGAGACTGTTA
GAGAACTCGAAGAAATAGGAAAAAGATGATCAAGCTGGGTGTTCCACGTGACACTCGTCAGTTCCAAAGT
CCCCAGATAGATCGTTCCCTATTTTTGCCACATTCTTTCTTTCTTTTTTCATTTAATTCCCCAAATCTTT
CATGTTTATT (SEQ ID NO:84)
Translation:
MQLYTYLYLLVPLVIFYLILGTGTLGHGGTLTERRSADATALKPEPVLLQKSAARSTGDDAKERLTQTKR
IRKKRANTTRGKEEDREIVETVRELEEIGKR (SEQ ID NO:85)
Toxin Sequence:
Gly-Lys-Xaa1-Xaa1-Asp-Arg-Xaa1-Ile-Val-Xaa1-Thr-Val-Arg-Xaa1-Leu-Xaa1-
Xaa1-Ile-# (SEQ ID NO:86)

| | |
|---|---|
| Name: | Conotoxin-L3 |
| Species: | lynceus |
| Cloned: | Yes |

DNA Sequence:
GCGATGCAACTGTACACGTATCTGTATCTGCTGGTGCCCCTGGTGACCTTCCACCTAATCCTAGGCACGG
GCACACTAGATCATGGAGGCGCACTGACTGAACGCCGTTCGACTGACGCCATAGCACTGAAACCTGAGCC
TGTCCTCCTGCAGAAATCCTCTGCCCGCAGCACCGACGACAATGGCAACGACAGGTTGATTCAGATGAAG
AGGATTCTCAAAAAGCGAGGAAACAAAGCCAGAGGCGAAGAGGAAGTTGCAAAAATGGCGGCAGAGCTTA
CCAGAGAAGAAGCTGTAAAGGGGAAATGATAATCAAGTTGGGTGTTCCACGTGACACTCGTCAGTTCTAA
AGTCCCCAGATAGATCGTTCCCTATTTTTGCCACATTCTTTCTTTCTATTTTCATTTAATTCCCCAAATC
TTTCATGTTTATT (SEQ ID NO:87)
Translation:
MQLYTYLYLLVPLVTFHLILGTGTLDHGGALTERRSTDAIALKPEPVLLQKSSARSTDDNGNDRLIQMKR
ILKKRGNKARGEEEVAKMAAELTREEAVKGK SEQ ID NO:88)
Toxin Sequence:
Gly-Xaa1-Xaa1-Xaa1-Val-Ala-Lys-Met-Ala-Ala-Xaa1-Leu-Thr-Arg-Xaa1-Xaa1-
Ala-Val-Lys-# (SEQ ID NO:89)

| | |
|---|---|
| Name: | Conotoxin-Fi1 |
| Species: | figulinus |
| Cloned: | Yes |

DNA Sequence:
GCGATGCAACTGTACACGTATCTGTATCTGCTGGTGCCCCTGGTGACCTTCTACCTAATCCTAGGCACGG
GCACGCTAGGTCATGGAGGCGCACTGACTGAACGCCGTTTGGCTGACGCCACAGCGCTGAAACCTGAGCC
TGTCCTCCTGCAGAAATCCGCTGCCCGCAGCACCGACGACAATGACAAGGACAGGCTGACCCAGATGAAG
AGGATTTTCAAAAAGCGAGGAAACAAAGCCAGAGGCGAGGAAGAAGTTGCAGAGATGGCGGCAGAGATTG
CAAGAGAAAATCAAGCAAACGGGAAAAGATAATCAAACTGGGTGTTCCACGTGACACTCGTCAGTTCTAA
AGTCCCCAGATAGGTCGTTCTCTATGTTTGCCACATTCTTTCTTTTTCTTTTCATTTAATTCCCCAAATC
TTTCATGTTTATT (SEQ ID NO:90)
Translation:
MQLYTYLYLLVPLVTFYLILGTGTLGHGGALTERRLADATALKPEPVLLQKSAARSTDDNDKDRLTQMKR
IFKKRGNKARGEEEVAEMAAEIARENQANGKR (SEQ ID NO:91)
Toxin Sequence:
Gly-Xaa1-Xaa1-Xaa1-Val-Ala-Xaa1-Met-Ala-Ala-Xaa1-Ile-Ala-Arg-Xaa1-Asn-
Gln-Ala-Asn-# (SEQ ID NO:92)

| | |
|---|---|
| Name: | Conotoxin-Fi2 |
| Species: | figulinus |
| Cloned: | Yes |

DNA Sequence:
GCGATGCAACTGTACACGTATCTGTATCTGCTGGTGCCCCTGGTGACCTTCTACCTAATCCTAGGGACGG
GCACACTAGCTCATGGAGGCGCACCGACTGAACGCCGTTTGGCTGACAGCACAGCACTGAAACCCGAGCA
TGTCCTCGTGCAGATGTCCGCTGCCCGCAGCACCAACGATAATGGCAAGGACAGGTTGACTCAGATGAAG
AGGATTCTCAAAAAGCAAGGAAACACAGGCAGAAGCTACGAACAAGCTAGAGAAGTTCAGGAGGCTGTTA
ATGAACTCAAGGAAAGAGGTAAAAAGATAATCATGCTGGGTGTTCCACGTGACACTCGTGAGTTGTAAAG
CCCCCAGATAGATTGTTGCGTATTTTTACCACGTTGTTTCTTTCTCTTTTCATTTAATTCCGGAAATCTT
TCATGTTTATT (SEQ ID NO:93)
Translation:
MQLYTYLYLLVPLVTFYLILGTGTLAHGGAPTERRLADTTALKPEHVLLQMSAARSTNDNGKDRLTQMKR
ILKKQGNTARSYEQAREVQEAVNELKERGKKIIMLGVPRDTRQF (SEQ ID NO:94)
Toxin Sequence:
Ser-Xaa5-Xaa1-Gln-Ala-Arg-Xaa1-Val-Gln-Xaa1-Ala-Val-Asn-Xaa1-Leu-Lys-
Xaa1-Arg-# (SEQ ID NO:95)

| | |
|---|---|
| Name: | Conotoxin-Fi2a |
| Species: | figulinus |
| Cloned: | Yes |

DNA Sequence:
GCGATGCAACTGTACACGTATCTGTATCTGCTGGTGCCCCTGGTGACCTTCTACCTAATCCTAGGGACGG
GCACACTAGCTGATGGAGGCGCACCGACTGAACGCCGTTTGGCTGACAGCACAGCACTGAAACGCGAGCA
TGTCCTCGTGCAGATGTCCGCTGCCCGCAGCACCAACGATAATGGCAAGGACAGGTTGACTCAGATGAAG
AGGATTCTCAAAAAGCAAGGAAACACAGCCAGAAGCTACGAACAAGCTAGAGAAGTTCAGGAGGCTGTTA
ATGAAGTCAAGGAAAGAGGTAAAAAGATAATCATGCTGGGTGTTCCACGTGACACTGGTCAGTTCTAAAG
CCCCCAGATAGATTGTTCCGTATTTTTACCACGTTCTTTCTTTCTCTTTTCATTTAATTCCCCAAATCTT
TCATGTTTATT (SEQ ID NO:96)
Translation:
MQLYTYLYLLVPLVTFYLILGTGTLAHGGAPTERRLADTTALKPEHVLLQMSAARSTNDNGKDRLTQMKR
ILKKQGNTARSYEQAREVQEAVNELKERGKKIIMLGVPRDTRQF (SEQ ID NO:97)
Toxin Sequence:
Ser-Xaa5-Xaa1-Gln-Ala-Arg-Xaa1-Val-Gln-Xaa1-Ala-Val-Asn-Xaa1-Leu-Lys-
Xaa1-Arg-Gly-Lys-Lys-Ile-Ile-Met-Leu-Gly-Val-Xaa3-Arg-Asp-Thr-Arg-Gln-
Phe-\hat{ } (SEQ ID NO:98)

| | |
|---|---|
| Name: | Conotoxin-Fi3 |
| Species: | figulinus |
| Cloned: | Yes |

DNA Sequence:
GCGATGCAACTGTACACGTATCTGTATCTGCTGGtGCCCCTGGTGAGGTTCCACCTAATCCTAGGCACGG
GCAGACTAGCTCATGGAGGCGCACTGGGTGAACGCCGTTTGGCTGACGCGACAGCGCTGAAAGCTGAGCC
TGTGGTCCTGCAGAAATGGGCTGCCCGCAGGAGCGAGGACAATGGCAAGGACAGGTTGAGTGAGATGAAG
AGGATTCTCAAAAAGCGAGGAAACACGGGCAGAGAGTAGGAAGATGATAGAGAGATTGGAGAGACTGTTA
GAGAACTCGAAGAAATAGGTAAAAGATAATGAAGCTGGGTGTTCAATTGAGACTCATCAGTTCTAAAGTC
CCCAGATAGATCGTTCCCTAATTTTGCCAGGTTCTTTCTTTCTCTTTTCATTTAATTCCCCAAATGTTTG
ATGTTTATT (SEQ ID NO:99)
Translation:
MQLYTYLYLLVPLVTFHLILGTGTLAHGGALAERRLADATALKPEPVLLQKSAARSTDDNGKDRLTEMKR
ILKKRGNTARDYEDDREIAETVRELEEIGKR (SEQ ID NO:100)
Toxin Sequence:
Asp-Xaa5-Xaa1-Asp-Asp-Arg-Xaa1-Ile-Ala-Xaa1-Thr-Val-Arg-Xaa1-Leu-Xaa1-
Xaa1-Ile-# (SEQ ID NO:101)

| | |
|---|---|
| Name: | Conotoxin-Fi4 |
| Species: | figulinus |
| Cloned: | Yes |

DNA Sequence:
GCGATGCAACTGTACACGTATCTGTATCTGCTGGTGCCCCTGGTGACCTTCTACCTAATGCTAGGCACGG
GCACGCTAGGTCATGGAGGCGCACTGACTGAACGCCGTTTGGGTGACGCCACAGCGCTGAAAGGTGAGCC
TGTCGTGCTGCAGAAATCCGCTGCCCGCAGCACCGACGACAATGGCAAGGACAGGTTGACTCAGATGAAG
GGGACTGTCAAAAAGCGAGGAAACACGGCCGAAGAAGTTAGAGAGGCTGCAGAGACTCTTCATGAACTCT
CGCTGTAGGAAAAAGAAAAAGATTAATCAAGCTGGGTGTTCCACGTGACACTCGTCAGTTCTAAAGTCCC
CAGTTCGCTATCTTTGCCACGTTTTTTCTTTCTCTTTTCATCCAATTCCCCAAATCTTTCATGTTTATT
(SEQ ID NO:102)
Translation:
MQLYTYLYLLVPLVTFYLILGTGTLGHGGALTERRLADATALKPEPVLLQKSAARSTDDNGKDRLTQMKG
TVKKRGNTAEEVREAAETLHELSL (SEQ ID NO:103)
Toxin Sequence:
Gly-Asn-Thr-Ala-Xaa1-Xaa1-Val-Arg-Xaa1-Ala-Ala-Xaa1-Thr-Leu-His-Xaa1-
Leu-Ser-Leu-\hat{ } (SEQ ID NO:104)

| | |
|---|---|
| Name: | Conotoxin-Fi5 |
| Species: | figulinus |
| Cloned: | Yes |

DNA Sequence:
GCGATGCAACTGTACACGTATCTGTATCTGCTGGTGCCTCTGGTGACCTTCCACCTAATCCTAGGCACGG
GCACACTAGGTCATGGAGGCGCACTGACTGAACGCCGTTTGGCTGACGCCACAGCGCTGAAACCTGAGCC
TGTCCTCCTGCAGAAATCCGCTGCCCGCAGCACCGACGTCAATGGCAAGGACAGGTTGACTGAGATGAAG
AGGATTCTCAAAAAGCGAGGAAGCATATCCATGGGCTTCGAACATAGAAGAGAGATTGCAGAGTTGGTTA
GAGAACTCGCTGAAATAGGTAAACGATAATCAAGCTGGGTGTTCCACTAACACTCGTCAGTTCTAAAGTC
CCCAGATAGATCGTTCCCTATCTTTGCCACATTTTTTTTCTCTTTTCATTTAATTCCCCAAATCTTTCAT
GTTTATT (SEQ ID NO:105)
Translation:
MQLYTYLYLLVPLVTFHLILGTGTLGHGGALTERRLADATALKPEPVLLQKSAARSTDVNGKDRLTEMKR
ILKKRGSISMGFEHRREIAELVRELAEIGKR (SEQ ID NO:106)
Toxin Sequence:
Gly-Ser-Ile-Ser-Met-Gly-Phe-Xaa1-His-Arg-Arg-Xaa1-Ile-Ala-Xaa1-Leu-
Val-Arg-Xaa1-Leu-Ala-Xaa1-Ile-# (SEQ ID NO:107)

| | |
|---|---|
| Name: | Conotoxin-Di1 |
| Species: | distans |
| Cloned: | Yes |

DNA Sequence:
GCGATGCAACTGTACACGTATCTGTATCTGCTGGTGCCCCTGGTGGCCTTCCACCTAATCCAAGGCACGG
GCACACTAGGCCATGGAGGCGCACTGACTGAAGGCCGTTCGGCTGACGCCACAGCGCCGAAACCTGAACC
TGTCCTCCTGCAGAAATCCGATGCCCGCAGCGCCGACGACAACGGCAAGGACAAGTTGACTCAGATGAAG
AGGACTCTGAAAAAGCAAGGACACATTGCCAGAACCATAACTGCTGAAGAGGCAGAGAGGACTAGTGAAA
GAATGTCATCAATGGGAAAAAGATAATCAAGCTGGGTGTTCCACGTGACACTCGTCAGTTCTAAAGTCCC
CAGATAAATCGTTCCCTGTTTTTGCCCTGTTCTTTCTTTCTCTTTTCATTCAATTCCCCAAATCTTTCAT
GTTTATT (SEQ ID NO:108)
Translation:
MQLYTYLYLLVPLVAFHLIQGTGTLGHGGALTEGRSADATAPKPEPVLLQKSDARSADDNGKDKLTQMKR
TLKKQGHIARTITAEEAERTSERMSSMGKR (SEQ ID NO:109)
Toxin Sequence:
Thr-Ile-Thr-Ala-Xaa1-Xaa1-Ala-Xaa1-Arg-Thr-Ser-Xaa1-Arg-Met-Ser-Ser-
Met-# (SEQ ID NO:110)

| | |
|---|---|
| Name: | Conotoxin-Di2 |
| Species: | distans |
| Cloned: | Yes |

DNA Sequence:
GCGATGCAACTGTACACGTATCTGTATCTGCTGGTATCCCTGGTGGCCTTCCACCTAATCCAAGGAACGG
GCACGCTAGGCCATGGAGGCGCACTGACTGAAGGCCGTTCGGCTGACGCCACAGCGCGGAAACCTGAACC
TGTGCTCGTGCAGAAATCGGATGCCCGCAGCGCCGACGACAACCGCAAGGACAAGTTGACTCAGATGAAG
AGGATTCTGAAAAAGCAAGAAACGCCAACTCCTGAAGAGGTAGAGCGCCATACCGAAAGACTCAAAAGCA
TGGGAAAAAGATAATCAAGCTGGGTGTTCCACGTGACACTCGTCAGTTCTAAAGTCCCCAGATGGATGGT
TGCCTGTTTTTGCCCCGTTCTTTCGTTCTCTTTTCATTCAATTCCCCAAATCTTTCATGTTTATT (SEQ
ID NO:111)
Translation:
MQLYTYLYLLVSLVAFHLIQGTGTLGHGGALTEGRSADATAPKPEPVLVQKSDARSADDNRKDKLTQMKR
ILKKQETPTPEEVERHTERLKSMGKR (SEQ ID NO:112)
Toxin Sequence:
Xaa2-Xaa1-Thr-Xaa3-Thr-Xaa3-Xaa1-Xaa1-Val-Xaa1-Arg-His-Thr-Xaa1-Arg-
Leu-Lys-Ser-Met-# (SEQ ID NO:113)

| | |
|---|---|
| Name: | Conotoxin-P1 |
| Species: | purpurascens |
| Cloned: | Yes |

DNA Sequence:
GCGATGCAACTGTACACGTATCTGTATCTGCTGGTGCCCCTGGTGACCTTCCACCTAATCCTAGGCACGG
GAATGCTAGCTCATGGAGACACACTGACTGAACGCCGTTCGGTTGACGCCAGAGCACTGAAACCTGAGCC
TGTCCTCCTGCAGAAATCCGCTGCCCGCAGCACCGACGACAATGACAAGGACAGGTTGACTCAGATGAAG
AGGATTCTCAAAAAGCGAGGAAACAAAGCCAGAGGCGAAGAAGAACATTCCAAGTATCAAGAGTGTCTTA
GAGAAGTAAGAGTAAATAAGGTACAACAAGAATGTTAATCAAGCTGGGTGTTCCACGTGACACTCGTCAG
TTCTAAAGTCCCCAGATAGATCGTTCCCGATTTTTGCCACATTCTTTCTTTCTCTTTTCATTTAATTCCC
CAAATCTTTGATGTTTATT (SEQ ID NO:114)
Translation:
MQLYTYLYLLVPLVTFHLILGTGMLAHGDTLTERRSVDATALKPEPVLLQKSAARSTDDNDKDRLTQMKR
ILKKRGNKARGEEEHSKYQECLREVRVNKVQQEC (SEQ ID NO:115)
Toxin Sequence:
Gly-Xaa1-Xaa1-Xaa1-His-Ser-Lys-Xaa5-Gln-Xaa1-Cys-Leu-Arg-Xaa1-Val-Arg-
Val-Asn-Lys-Val-Gln-Gln-Xaa1-Cys-\hat{ } (SEQ ID NO:116)

| | |
|---|---|
| Name: | Conotoxin-P2 |
| Species: | purpurascens |
| Cloned: | Yes |

DNA Sequence:
GCGATGCAAGTGTACACGTATCTGTATCTGCTGGTGCCCCTGGTGACCTTCCACCTAATCCTAGGCACGG
GCACACTAGCTCATGGAGGCGCACTGACTGAACGCGGTTCCACTGACGCCACAGCACTGAAACCTGAGCC
TGTCCTGCAGGAATCTGATGCCCGCAGCACCGACGACAATGACAAGGACAGGTTGACTCAGATGAAGAGG
ATTCTCAAAAAGCGAGGAAACAAAGCCAGAGGCGAAGAAGAACATTCCAAGTATCAGGAGTGTCTTAGAG
AAGTAAGAGTAAATAACGTACAAGAAGAATGTTAATCAAGCTGGGTGTTCCACGTGACACTCGTCAGTTC
TAAAGTCCCCAGATAGATCGTTCCCTATTTTTGCCACATTCTTTCTTTCTCTTTTCATTTAATTCCCCAA
ATCTTTCATGTTTATT (SEQ ID NO:117)
Translation:
MQLYTYLYLLVPLVTFHLILGTGTLAHGGALTERGSTDATALKPEPVLQESDARSTDDNDKDRLTQMKRI
LKKRGNKARGEEEHSKYQECLREVRVNNVQQEG (SEQ ID NO:118)
Toxin Sequence:
Gly-Xaa1-Xaa1-Xaa1-His-Ser-Lys-Xaa5-Gln-Xaa1-Cys-Leu-Arg-Xaa1-Val-Arg-
Val-Asn-Asn-Val-Gln-Gln-Xaa1-Cys-\hat{ } (SEQ ID NO:119)

| | |
|---|---|
| Name: | Conotoxin-P3 |
| Species: | purpurascens |
| Cloned: | Yes |

DNA Sequence:
GCGATGCAACTGTACACGTATCTGTATCTGCTGGTGCCCCTGGTGACCTTCCACCTAATCCTAAGCACGG
GCACACTAGCTCATGGAGGCACACTGACTGAACGCCGTTCGACTGACACCACAGCACTGAAACCTGAGCC
TGTCCTCCTGCAGAAATCTGATGCCCGCAGCACCGACGACAATGACAAGGACAGGTTGACTCAGATGAAG
AGGATTCTCAAAAAGCGAGGAAACAAAGCCAGAGGCGAAGAAGAACATTCCAAGTATCAGGAGTGTCTTA
GAGAAATAAGAGTAAATAAGGTACAACAAGAATGTTAATCAAGCTGGGTGTTCCACGTGACACCCGTCAG
TTCTAAAGTCCCCAGATAGATCGTTCCCTATTTTTGCCACATTCTTTCTTTCTCTTTTCATTTAATTCCC
CAAATCTTTCATGTTTATT (SEQ ID NO:120)
Translation:
MQLYTYLYLLVPLVTFHLILSTGTLAHGGTLTERRSTDTTALKPEPVLLQKSDARSTDDNDKDRLTQMKR
ILKKRGNKARGEEEHSKYQECLREIRVNKVQQEC (SEQ ID NO:121)
Toxin Sequence:
Gly-Xaa1-Xaa1-Xaa1-His-Ser-Lys-Xaa5-Gln-Xaa1-Cys-Leu-Arg-Xaa1-Ile-Arg-
Val-Asn-Lys-Val-Gln-Gln-Xaa1-Cys-\hat{ } (SEQ ID NO:122)

| | |
|---|---|
| Name: | Conotoxin-P4 |
| Species: | purpurascens |
| Cloned: | Yes |

DNA Sequence:
GCGATGCAACTGTACACGTATCTGTATCTGCTGGTGCCCCTGGTGACCTTCCACCTAATCCTAAGGACGG
GCACACTAGCTCATGGAGACACACTGACTGAACGCGGTTCGGTTGACGCCACAGCACTGAAACCTGAGCC
TGTCCTCCTGCAGAAATCCGCTGCCCGCAGCACCGACGACGATGACAAGGACAGGTTGACTCAGAGGAAG
AGGATTCTCAAAAAGGAAGCAAACAAAGCCAGAGGCGAAGCAGAACATTACGCGTTTCAGGAGTGTCTTA
GAGAAATAAATGTAAATAAGGTACAACAAGAATGTTAATCAAGCTGGGTGTTCTACGTGACACTCGTCAG
TTCTAAAGTCCCCAGATAGATCGTTCGCTATTTTTGCCACATTCTTTCTTTCTCTTTTCATTTAATTCCC
CAAATCTTTCATGTTTATT (SEQ ID NO:123)
Translation:
MQLYTYLYLLVPLVTFHLILSTGTLAHGDTLTERRSVDATALKPEPVLLQKSAARSTDDDDKDRLTQRKR
ILKKQGNKARGEAEHYAFQEGLREINVNKVQQEG (SEQ ID NO:124)
Toxin Sequence:
Gly-Xaa1-Ala-Xaa1-His-Xaa5-Ala-Phe-Gln-Xaa1-Cys-Leu-Arg-Xaa1-Ile-Asn-
Val-Asn-Lys-Val-Gln-Gln-Xaa1-Cys-\hat{ } (SEQ ID NO:125)

| | |
|---|---|
| Name: | Conotoxin-P5 |
| Species: | purpurascens |
| Cloned: | Yes |

DNA Sequence:
GCGATGCAACTGTACACGTATCTGTATCTGCTGGTGCCCCTGGTGACCTTCCACCTAATCCTAGGCACGG
GAATGCTAGCTCATGGAGACACACTGACTGAACGCCGTTCGGTTGACGCCACAGCACTGAAACCTGAGCC
TGTCCTCCTGCAGAAATCCGCTGCCCGCAGCACCGACGCCAATGGCAAGGACAGGTTGACTCAGAGGAAG
AGGATTCTCAAAAAGCGAGGAAACATGGCCAGGGGCTTAGAAGAAGATATAGAGTTTATTGAGACGATCG
AAGAAATTGGAAAAAGATAACCAAGCTGGGTGTTCCACGTGACACTCGTCGGTTCTAAAGTCCCCAGATA
GATCGTTCACTATTTTTGCCACATTCTTTCTTTCTCTTTTCATTTAATTCCCCAAATCTTTCATGTTTAT
T (SEQ ID NO:126)
Translation:
MQLYTYLYLLVPLVTFHLILGTGMLAHGDTLTERRSVDATALKPEPVLLQKSAARSTDANGKDRLTQRKR
ILKKRGNMARGLEEDIEFIETIEEIGKR (SEQ ID NO:127)
Toxin Sequence:
Gly-Leu-Xaa1-Xaa1-Asp-Ile-Xaa1-phe-Ile-Xaa1-Thr-Ile-Xaa1-Xaa1-Ile-#
(SEQ ID NO:128)

| | |
|---|---|
| Name: | Conotoxin-Sm1 |
| Species: | stercusmuscarum |
| Cloned: | Yes |

DNA Sequence:
GCGATGCAACTGTACACGTATCTGTATCTGCTGGTGCCCCTGGTGACCTTCCACCTAATCCTGGGCACGG
GCACACTAGATCATGGAGGCGCACTGACTGAACGCCGTTCGGCTGACGCGACAGCGCTGAAACCTGAGCC
TGTCCTGCAGAAATCCGCTGCCGGCAGCACCGACGACAACGGCAAGGACAGGTTGACTCAGATGAAGAGG
ATTCTCAAAAAGCGAGGAAACACGGCTAGAATCACCGAAACTGATATAGAGCTTGTTATGAAATTAGAAG
AAATTGGAAAAAGATAATCAAGCTGGGTGTTCCACGTGACAGTCGTGAGTTCTGAAGTCCCGAGGTAGAT
CGTTCCCTATTTTTGCCACATTCTTTCTTTCTCTTTTCATGTAATTCCCCAAATCTTTCATGTTTATT
(SEQ ID NO:129)
Translation:
MQLYTYLYLLVPLVTFHLILGTGTLDHGGALTERRSADATALKPEPVLQKSAAGSTDDNGKDRLTQMKRI
LKKRGNTARITETDIELVMKLEEIGKR (SEQ ID NO:130)
Toxin Sequence:
Ile-Thr-Xaa1-Thr-Asp-Ile-Xaa1-Leu-Val-Met-Lys-Leu-Xaa1-Xaa1-Ile-# (SEQ
ID NO:131)

Where:
Xaa1 = Glu or γ-Carboxy Glu
Xaa2 = Gln or pyroglu
Xaa3 = Pro or Hydroxy Pro
Xaa4 = Trp (D or L) or Bromo Trp (D or L)
Xaa5 = Tyr, ¹²⁵I-Tyr, Mono-Iodo Tyr, Di-Iodo Tyr, O-sulpho-Tyr or O-Phospho-Tyr
\hat{ } = Free-carboxyl C-term or Amidated C-term, preferably Free-carboxyl
# = Free-carboxyl C-term or Amidated C-term, preferably Amidated
? = Status of C-term not known.

TABLE 5

Alignment of Linear γ-Carboxyglutamate Rich Conotoxins
(SEQ ID NO:) With Respect to Conantokin G

| | | |
|---|---|---|
| Conantokin G | -----GEXXL-QX-NQXLIRX-KSN# | (SEQ ID NO:132) |
| Conotoxin-Af6 | ZGQDDSEXXDSQKVMKHGQRRERR\hat{ } | (SEQ ID NO:133) |
| Conotoxin-Bt1 | -----GGXXV-RX-SAXTLHXLTP\hat{ } | (SEQ ID NO:134) |
| Conotoxin-Bt2 | -----GGXXV-RX-SAXTLHXITP\hat{ } | (SEQ ID NO:135) |
| Conotoxin-Bt3 | -----DGXXV-RX-AAXTLNXLTP\hat{ } | (SEQ ID NO:136) |
| Conotoxin-Bt4 | -----GY-XDDRX-IAXTVRXLEEA# | (SEQ ID NO:137) |
| Conotoxin-Bt5 | -----GGGXV-RX-SAXTLHXITP\hat{ } | (SEQ ID NO:138) |
| Conotoxin-Bu1 | -----NP-XTYIX-IVXISRXLEEI# | (SEQ ID NO:139) |
| Conotoxin-Bu2 | -----NP-XTYY--NLX-LVXISRELEEI# | (SEQ ID NO:140) |
| Conotoxin-C1 | -----SDXXLLRX-DVXTVLXLERN# | (SEQ ID NO:141) |
| Conotoxin-C2 | -----GDXXLLRX-DVXTVLXLERD# | (SEQ ID NO:142) |
| Conotoxin-C3 | -----SDXXLLRX-DVXTVLXPERN# | (SEQ ID NO:143) |
| Conotoxin-C4 | -----IE-XGLIX-DLXTARXRDS# | (SEQ ID NO:144) |
| Conotoxin-C5 | -----IE-XGLIX-DLXAARXRDS# | (SEQ ID NO:145) |
| Conotoxin-C6 | -----GEPXVGS--IPXAVRQQECIRNNNNRPWCPK\hat{ } | (SEQ ID NO:146) |
| Conotoxin-Di1 | -T--ITAXXA--XRTSXRMSSM# | (SEQ ID NO:147) |
| Conotoxin-Di2 | ZET-PTPXXV--XRHTXRLKSM# | (SEQ ID NO:148) |
| Conotoxin-Ep1 | G--GKDIVXTITX--LXKI# | (SEQ ID NO:149) |
| Conotoxin-Fi1 | -----GEXXV-AXMAAXIARXNQAN# | (SEQ ID NO:150) |
| Conotoxin-Fi2 | -----S-YXQARX-VQXAVNXLKER# | (SEQ ID NO:151) |
| Conotoxin-Fi2a | -----S-YXQARX-VQXAVNXLKERGKKIIMLGVPRDTRQFA\hat{ } | (SEQ ID NO:152) |
| Conotoxin-Fi3 | -----D-YXDDRX-IAXTVRXLEEI# | (SEQ ID NO:153) |
| Conotoxin-Fi4 | GNTA---XXV-RX-AAXTLHELS-L\hat{ } | (SEQ ID NO:154) |
| Conotoxin-Fi5 | GSISMG-FXHRRX-IAXLVRELAEI# | (SEQ ID NO:155) |
| Conotoxin-L1 | ----GEXXVAK-MAAXIARXNAAN# | (SEQ ID NO:156) |
| Conotoxin-L2 | -----GKXXD-RX-IVXTVRXLEEI# | (SEQ ID NO:157) |
| Conotoxin-L3 | -----GEXXVAK-MAAXLTRXEAVK# | (SEQ ID NO:158) |
| Conotoxin-P1 | -----GEXXHSK--YQXCLRXVRVNKVQQEC\hat{ } | (SEQ ID NO:159) |
| Conotoxin-P2 | -----GEXXHSK--YQXCLRXVRVNNVQQEC\hat{ } | (SEQ ID NO:160) |
| Conotoxin-P3 | -----GEXXHSK--YQXCLRXIRVNKVQQEC\hat{ } | (SEQ ID NO:161) |
| Conotoxin-P4 | -----GEAXHYA--FQXCLRXINVNKVQQEC\hat{ } | (SEQ ID NO:162) |
| Conotoxin-P5 | -----GLXXD-IX-FIX-TIXEI# | (SEQ ID NO:163) |
| Conotoxin-Sm1 | -----IT-XTDIXLVMKL--XEI# (SEQ ID NO:164) | |

X is Glu or Gla, preferably Gla particularly with respect to positions 3 and 4 in conantokin G

TABLE 6
Alignment of Linear γ-Carboxyglutamate Rich Conotoxins¹

| | | |
|---|---|---|
| Conotoxin-Af6 | ZGQDDSEEEDSQKVMKHGQRRERR\hat{ } | (SEQ ID NO:165) |
| Conotoxin-Bt1 | G--G----EEVRESAETLHELT-P\hat{ } | (SEQ ID NO:166) |
| Conotoxin-Bt2 | G--G----EEVRESAETLHEIT-P\hat{ } | (SEQ ID NO:167) |
| Conotoxin-Bt3 | D--G----EEVREAAETLNELT-P\hat{ } | (SEQ ID NO:168) |
| Conotoxin-Bt4 | G------YEDDREIAETVRELEEA# | (SEQ ID NO:169) |
| Conotoxin-Bt5 | G--G----GEVRESAETLHEIT-P\hat{ } | (SEQ ID NO:170) |
| Gonotoxin-Bu1 | NPETY------IEIVEISRELEEI# | (SEQ ID NO:171) |
| Conotoxin-Bu2 | NPETY----YNLELVEISRELEEI# | (SEQ ID NO:172) |
| Conotoxin-C1 | SDEEL-----LREDVETVLELERN# | (SEQ ID NO:173) |
| Conotoxin-C2 | GDEEL-----LREDVETVLELERD# | (SEQ ID NO:174) |
| Conotoxin-C3 | SDEEL-----LREDVETVLEPERN# | (SEQ ID NO:175) |
| Conotoxin-C4 | IEEGL-----I-EDLETARERD-S# | (SEQ ID NO:176) |
| Conotoxin-C5 | IEEGL-----I-EDLEAARERD-S# | (SEQ ID NO:177) |
| Conotoxin-C6 | GEPEVGSIPEAVRQQECIRNNNNRPWCPK\hat{ } | (SEQ ID NO:178) |
| Conotoxin-Di1 | --T--ITAEEAERTSERMSSM# | (SEQ ID NO:179) |
| Conotoxin-Di2 | ZET--PTPEEVERHTERLKSM# | (SEQ ID NO:180) |
| Conotoxin-Ep1 | G--G-------KDIVETITELEKI# | (SEQ ID NO:181) |
| Conotoxin-Fi1 | GEEEVAE-----MAAEIARENQAN# | (SEQ ID NO:182) |
| Conotoxin-Fi2 | -----S-YEQAREVQEAVNELKER# | (SEQ ID NO:183) |
| Conotoxin-Fi2a | -----S-YXQAREVQEAVNELKERGKKIIMLGVPRDTRQF\hat{ } | (SEQ ID NO:184) |
| Conotoxin-Fi3 | D------YEDDREIAETVRELEEI# | (SEQ ID NO:185) |
| Conotoxin-Fi4 | GNTA----EEVREAAETLHELS-L\hat{ } | (SEQ ID NO:186) |
| Conotoxin-Fi5 | GSISMG-FEHRREIAELVRELAEI# | (SEQ ID NO:187) |
| Conotoxin-L1 | GEEEVAK-----MAAEIARENAAN# | (SEQ ID NO:188) |
| Conotoxin-L2 | G------KEEDREIVETVRELEEI# | (SEQ ID NO:189) |
| Conotoxin-L3 | GEEEVAK-----MAAELTREEAVK# | (SEQ ID NO:190) |
| Conotoxin-P1 | GEEEHSKYQECLREVRVNKVQQEC\hat{ } | (SEQ ID NO:191) |
| Conotoxin-P2 | GEEEHSKYQECLREVRVNNVQQEC\hat{ } | (SEQ ID NO:192) |
| Conotoxin-P3 | GEEEHSKYQECLREIRVNKVQQEC\hat{ } | (SEQ ID NO:193) |
| Conotoxin-P4 | GEAEHYAFQECLREINVNKVQQEC\hat{ } | (SEQ ID NO:194) |
| Conotoxin-P5 | G---LEEDIEFIETIE------EI# | (SEQ ID NO:195) |
| Conotoxin-Sm1 | ------ITETDIELVMKL----EEI# | (SEQ ID NO:196) |

¹The sequences are compared prior to γ-carboxylation.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Abiko, H. et al. (1986). Brain Res. 38:328-335.
Aldrete, J. A. et al. (1979). Crit. Care Med. 7:466-470.
Bliss, et al. (1993). Nature 361:31.
Bormnann, J. (1989). Euro. J. Pharmacol. 166:591-592.
Brauner-Osborne, H. et al. (2000). J. Medicinal Chem. 43:2609-2645.
Chandler, P. et al. (1993). J. Biol. Chem. 268:17173-17178.
Chaplan S. R. (1997). J. Pharmacol. Exp. Ther. 280:829-838.
Codere, T. J. (1993). Eur. J. Neurosci. 5:390-393.
Ettinger, L. J. et al. (1978). Cancer 41:1270-1273.
Greenamyre, J. T. and O'Brien, C. F. (1991). Arch. Neurol. 48:977-981.
Heyes, M. P., et al. (1989). Ann. Neurol. 26: 275-277.
Johnson et al. (1990). Ann. Rev. Pharmacol. Toxicol. 30:707-750.
Luer, M. S. & Hatton, J. (1993). Annals Pharmcotherapy 27:912-921.
Lipton, S. A. (1991). Neuron 7:111-118.
Lipton, S. A. (1994). Dav Neurosci. 61:145-151.
Lipton, S. A. (1996). Brain Pathol 6:507-517.
Liu, H. et al. (1997). Nature 386:721-724.
McIntosh, J. M. et al. (1998). Methods Enzymol. 294:605-624.
The Merck Manual of Diagnosis and Therapy, 16 Ed., Berkow, R. et al., eds., Merck Research Laboratories, Rahway, N.J., pp. 1436-1445 (1992).
Muller, W. E. et al. (1996). Prog Mol Subcell Biol. 16:44-57.
Nehlig, A. et al. (1990). Effects of phenobarbital in the developing rat brain. In Neonatal Seizures, Wasterlain, C. G. and Vertt, P. (eds.), Raven Press, New York, pp. 285-194.
Nishida, K. et al. (1996). J Neurochem 66:433-435.
Olney, J. W. et al. (1987). Eur. J. Pharmacol. 142:319-320.
Olivera, B. M. et al. (1985). Science 230:1338-1343.
Olivera, B. M. et al. (1990). Science 249:257-263.
Olivera, B. M. et al. (1996). U.S. Pat. No. 5,514,774.
Ornstein, et al. (1993). Biorganic Medicinal Chemistry Letters 3:43-48.
Popik, P. et al. (1995). Pharmacol. Rev. 47:235-253.
Raber, J. et al. (1996). Virology 226:362-373.
Rall T. W. and Schleifer, L. S. in Goodman and Gilman's The Pharmacological Basis of Therapeutics, Seventh Ed., Gilman, A. G. et al., eds., Macmillan Publishing Co., New York, pp. 446-472 (1985).
Remington's Pharmaceutical Sciences, 18th Ed. (1990, Mack Publishing Co., Easton, Pa.).
Roberts et al. (1983). The Peptides 5:342-429.
Rytik, P. G. et al. (1991). Anti-HIV activity of memantine. AIDS Res Hum Retrovir 7:89-95.
Sambrook, J. et al. (1989). Molecular Cloning: A Laboratory Manual, 2nd Ed., Cold Spring Harbor Laboratory, Cold Spring Harbor, N.Y.
Sei, Y. et al. (1996). J. Neurochem. 66:296-302.
Skolnick, P. et al. (1992). J. Neurochem. 59:1526-1521.
Spanagel, R. and Zieglgansberger, W. (1997). Trends Pharmacol. Sci. 18:54-59.
Sweetman, P. M. (1993). Eur. J. Neurosci. 5:276-283.
Starr, M. S. (1995). J. Neural Tans.[P-D Sect] 10:141-185.
Troupin, A. S. et al. (1986). MK-801. In New Anticonvulsant Drugs, Current Problems in Epilepsy 4, Meldrum, B. S. and Porter, R. J. (eds.), John Libbey, London, pp. 191-202.
Trujillo, K. A. and Akil, H (1995). Drug Alcohol Depend. 38:139-154.
Ungerstedt, U. et al. (1973). Animal Models of Parkinsonism. In Advances in Neurology: Progress in the Treatment of Parkinsonism, Calne, D. B., Ed., Raven Press, N.Y., pp 257-271.
Van de Steen, P. et al. (1998). Critical Rev. in Biochem. and Mol. Biol. 33:151-208.
White, H. S., et al. (1992). Epilepsy Res. 12:217-226.
White, H. S., et al. (1995). Experimental Selection, Quantification, and Evaluation of Antiepileptic Drugs. In Antiepileptic Drugs, 4th Ed., Levy, R. H., eds., Raven Press, N.Y., pp. 99-110.
Wong, E. H. P. et al. (1986). Proc. Natl. Acad. Sci. USA 83:7104-7108.
Zigmond, M. J. et al. (1987). Parkinsonism: Insights from animal models utilizing neurotoxic agents. In Animal Models of Demential, Coyle, J. T., Ed., Alan R. Liss, Inc., pp 1-38.
Zimm, S. et al. (1984). Cancer Res. 44:1698-1701.
U.S. Pat. No. 5,550,050 (1996).
U.S. Pat. No. 5,844,077 (1998).
Published PCT Application WO 92/19195 (1992).
Published PCT Application WO 94/25503 (1994).
Published PCT Application WO 95/01203 (1995).
Published PCT Application WO 95/05452 (1995).
Published PCT Application WO 96/02286 (1996).
Published PCT Application WO 96/02646 (1996).
Published PCT Application WO 96/40871 (1996).
Published PCT Application WO 96/40959 (1996).
Published PCT Application WO 97/12635 (1997).
Published PCT Application WO 98/03189 (1998).
PCT Published Application WO 00/23092 (2000).

What is claimed is:

1. An isolated peptide selected from the group consisting of:

| | |
|---|---|
| Conotoxin-Af6: | X₆GQDDSX₁X₁X₁DSQX₂VMX₂HGQRRERR\hat{ } |
| Conotoxin-Bt1: | GGX₁X₁VRX₁SAX₁TLHX₁LTX₅\hat{ } |
| Conotoxin-Bt2: | GGX₁X₁VRX₁SAX₁TLHX₁ITX₅\hat{ } |
| Conotoxin-Bt3: | DGX₁X₁VRX₁AAX₁TLNX₁LTX₅\hat{ } |
| Conotoxin-Bt4: | GYX₁DDRX₁IAX₁TVRX₁LX₁X₁A# |
| Conotoxin-Bt5: | GGGX₁VRX₁SAX₁TLHX₁ITX₅\hat{ } |
| Conotoxin-Bu1: | NX₅X₁TX₃IX₁IVX₁ISRX₁LX₁X₁I# |
| Conotoxin-Bu2: | NX₅X₁TX₃X₃NLX₁LVX₁ISRX₁LX₁X₁I# |
| Conotoxin-C1: | SDX₁X₁LLRX₁DVX₁TVLX₁LX₁RN# |
| Conotoxin-C2: | GDX₁X₁LLRX₁DVX₁TVLX₁LX₁RD# |
| Conotoxin-C3: | SDX₁X₁LLRX₁DVX₁TVLX₁PX₁RN# |
| Conotoxin-C4: | IX₁X₁GLIX₁DLX₁TARX₁RDS# |
| Conotoxin-C5: | IX₁X₁GLIX₁DLX₁AARX₁RDS# |
| Conotoxin-CG: | GX₁X₅X₁VGSIX₅X₁AVRQQX₁CIRNNNNRX₅X₄CX₅X₂\hat{ } |
| Conotoxin-Di1: | TITAX₁X₁AX₁RTSX₁RMSSM# |
| Conotoxin-Di2: | X₆X₁TX₅TX₅X₁X₁VX₁RHTX₁RLKSM# |
| Conotoxin-Ep1: | GGKDIVX₁TITX₁LX₁X₂I# |
| Conotoxin-Fi1: | GX₁X₁X₁VAX₁MAAX₁IARX₁NQAN# |
| Conotoxin-Fi2: | SX₃X₁QARX₁VQX₁AVNX₁LX₂X₁R# |
| Conotoxin-Fi2a: | SX₃X₁QARX₁VQX₁AVNX₁LX₂X₁RGX₂X₂IIMLGVX₅RDTRQF\hat{ } |
| Conotoxin-Fi3: | D X₃X₁DDRX₁IAX₁TVRX₁LX₁X₁I# |
| Conotoxin-Fi4: | GNTAX₁X₁VRX₁AAX₁TLHX₁LSL\hat{ } |
| Conotoxin-Fi5: | GSISMGFX₁HRRX₁IAX₁LVRX₁LAX₁I# |
| Conotoxin-L1: | GX₁X₁X₁VAX₁MAAX₁IARX₁NAAN# |
| Conotoxin-L2: | GX₂X₁X₁DRX₁IVX₁TVRX₁LX₁X₁I# |
| Conotoxin-L3: | GX₁X₁X₁VAX₂MAAX₁LTRX₁X₁AVX₂# |
| Conotoxin-P1: | GX₁X₁X₁HSX₂X₃QX₁CLRX₁VRVNX₂VQQX₁C\hat{ } |
| Conotoxin-P2: | GX₁X₁X₁HSX₂X₃QX₁CLRX₁VRVNNVQQX₁C\hat{ } |
| Conotoxin-P3: | GX₁X₁X₁HSX₂X₃QX₁CLRX₁IRVNX₂VQQX₁C\hat{ } |
| Conotoxin-P4: | GX₁AX₁HX₃AFQX₁CLRX₁INVNX₂VQQX₁C\hat{ } |
| Conotoxin-P5: | GLX₁X₁DIX₁FIX₁TIX₁X₁I# |
| Conotoxin-Sm1: | ITX₁TDIX₁LVMX₂LX₁X₁I# |

wherein X₁ is Glu or γ-carboxyglutamic acid (Gla); X₂ is Lys, nor-Lys, N-methyl-Lys, N,N-dimethyl-Lys or N,N,N-trimethyl-Lys; X₃ is Tyr, mono-halo-Tyr, di-halo-Tyr, O-sulpho-Tyr, O-phospho-Tyr or nitro-Tyr; X₄ is Trp (D or L) or halo-Trp (D or L); X₅ is Pro or hydroxy-Pro; and X₆ is Gln or pyroglutamate.

2. A derivative of the peptide of claim 1, in which the Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; the Asn, Ser, Thr or Hyp residues may be glycosylated; the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid; and the aliphatic amino acids may be substituted by synthetic derivatives bearing non-natural aliphatic branched or linear side chains CₙH₂ₙ₊₂ up to and including n=8.

3. An isolated nucleic acid encoding a conopeptide propeptide having an amino acid sequence set forth in Table 4.

4. The isolated nucleic acid of claim 3, wherein the nucleic acid comprises a nucleotide sequence set forth in Table 4.

5. An isolated conopeptide propeptide having an amino acid sequence set forth in Table 4.

6. A method for treating or preventing disorders in which the pathophysiology involves excessive excitation of nerve cells by excitatory amino acids or agonists of heterogenous ionotropic glutamate receptors or heterogenous G protein coupled glutamate receptors which comprises administering to a patient in need thereof a therapeutically effective amount of the peptide of claim 1 or a pharmaceutically acceptible salt thereof.

7. The method of claim 6, wherein said disorder is a neurologic disorder or a psychiatric disorder.

8. The method of claim 7, wherein said neurologic disorder is a seizure.

9. The method of claim 8, wherein said seizure is seizure is associated with epilepsy.

10. The method of claim 7, wherein said neurologic disorder is a neurotoxic injury associated with conditions of hypoxia, anoxia or ischemia.

11. The method of claim 10, wherein said neurotoxic injury is associated with stroke, cerebrovascular accident, brain or spinal cord trauma, myocardial infarct, physical trauma, drownings, suffocation, perinatal asphyxia, or hypoglycemic events.

12. The method of claim 7, wherein said neurologic disorder is neurodegeneration.

13. The method of claim 12, wherein said neurodegeneration is associated with Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia from HIV infection, multi-infarct dementia, Binswanger dementia and neuronal damage associated with uncontrolled seizures.

14. The method of claim 13 wherein said treatment is for HIV infection.

15. The method of claim 7, wherein said neurologic disorder is pain.

16. The method of claim 15, wherein said pain is migraine, acute pain or persistent pain.

17. The method of claim 7, wherein said neurologic disorder is chemical toxicity.

18. The method of claim 17, wherein said chemical toxicity is addiction, morphine tolerance, opiate tolerance, opioid tolerance and barbiturate tolerance.

19. The method of claim 7, wherein said neurologic disorder is dystonia (movement disorder), urinary incontinence, muscle relaxation or sleep disorder.

20. The method of claim 19, wherein said disorder is urinary incontinence.

21. The method of claim 7, wherein said psychiatric disorder is anxiety, major depression, manic- depressive illness, obsessive-compulsive disorder, schizophrenia or mood disorder.

22. The method of claim 21, wherein said mood disorder is bipolar disorder, unipolar depression, dysthymia or seasonal effective disorder.

23. A method for treating memory or cognitive deficits, HIV infection, or ophthalmic indications which comprises administering to a patient in need thereof a therapeutically effective amount of the peptide of claim 1 or a pharmaceutically acceptible salt thereof.

24. A method for controlling nematodes or parasitic worms which comprises applying an effective amount of a peptide of claim 1 to the locus to be protected.
